(19) United States
(12) Patent Application Publication
(10) Pub. No.: US 2026/0191856 A1
(43) Pub. Date: Jul. 9, 2026

(54) ALLOSTERIC MODULATORS OF THE μ-OPIOID RECEPTOR AND CANNABINOID RECEPTOR 1

(71) Applicants: CZ BIOHUB SF, LLC, San Francisco, CA (US); THE BOARD TRUSTEES OF THE LELAND STANFORD JUNIOR UNIVERSITY, Stanford, CA (US)

(72) Inventors: Evan O'Brien, Stanford, CA (US); Kaavya Krishna Kumar, Stanford, CA (US); Brian Kobika, Stanford, CA (US)

(21) Appl. No.: 19/128,374

(22) PCT Filed: Nov. 21, 2023
(86) PCT No.: PCT/US2023/080637

§ 371 Date: May 8, 2025

Related U.S. Application Data
(60) Provisional application No. 63/426,812, filed on Nov. 21, 2022.
(60) Provisional application No. 63/537,298, filed on Sep. 8, 2023.

(51) Int. Cl. A61K 31/5025; A61K 31/165; A61K 31/4045; A61K 31/426; A61K 31/4418; A61P 25/36

(52) U.S. Cl. CPC A61K 31/5025; A61K 31/165; A61K 31/4045; A61K 31/426; A61K 31/4418; A61P 25/36

(57) ABSTRACT

Disclosed herein are methods of using compounds that allosterically modulate the μ-opioid receptor (MOD) or cannabinoid receptor 1 (CB1) to treat pain (for positive allosteric modulators) or to treat opioid overdoses (for negative allosteric modulators).

### FIELD

The present disclosure relates generally to methods for treating pain by modulating the μOR and CB1 receptors and treating opioid overdose by modulating the μOR.

### BACKGROUND

Traditional opioid compounds extracted from Papaver somniferum and related semi-synthetic derivates, including morphine, heroin, codeine, and oxycodone, have been used both recreationally and as potent pain relief molecules. These compounds represent best-in-class treatments for acute pain management¹ yet have also been extensively prescribed as long-term analgesic treatments, where their high potential for addiction and abuse have fueled the current opioid overdose epidemic¹⁻³. While ever more patients were becoming addicted to over-prescribed long-term pain management opioids (e.g., oxycodone), fully synthetic, much more potent opioids such as fentanyl have exploded as cheap additives to recreationally-used opioid mixtures^{4, 5}. Naloxone (Narcan) is the most common, effective treatment for opioid overdoses but requires larger, repeated doses in response to the more potent fentanyl^{5, 6}. All these molecules share a similar mode of action as agonists of the μ-opioid receptor (MOR), albeit with varying affinity and efficacy.

Agonists and antagonists of the MOR bind at an overlapping orthosteric site in the extracellular vestibule of the receptor and share a set of key interactions with endogenous opioid signaling peptides, namely the enkephalins, endorphins and endomorphins⁷⁻⁹. Given the limitations of the current slate of orthosteric opioid molecules, as well as the severity of the current illegal opioid overdose epidemic, new classes of MOR-modulating compounds with distinct mechanisms of action are highly desirable and have emerged as priorities of the National Institute of Drug Abuse (NIDA)¹⁰.

Cannabinoid receptors 1 and 2 (CB1 and CB2) are integral components of pain modulation³⁸, responding in the body to natural endocannabinoid (eCB) systems. There is substantial evidence that CB1 is a therapeutic target for pain. However, due to the widespread expression of CB1 in the central nervous system, orthosteric CB1 receptor agonists are unusable as therapeutics due to psychotropic and neurological side-effects²⁰. Additionally, due to high sequence identity between CB1 and CB2 in the orthosteric pocket, developing subtype selective orthosteric drugs is challenging. Moreover, synthetic orthosteric agonists for CB1 have been shown to have different signaling profiles when compared to eCBs²¹, and produce a tonic activation of receptors throughout the body, both of which contribute to their adverse effects. Studies have shown that ZCZ-011 (ZCZ), a positive allosteric modulator (PAM) of CB1 enhances the antinociceptive effects without displaying psychoactive side-effects²².

Thus, a need exists for modulators of MOR and CB1 and their use in treating pain and opioid overdose.

### SUMMARY

Provided herein are methods of positively modulating the μ-opioid receptor (MOR) comprising contacting MOR with a positive allosteric modulator (PAM) in an amount effective to positively modulate MOR, wherein the MOR-PAM is selected from the following compounds:

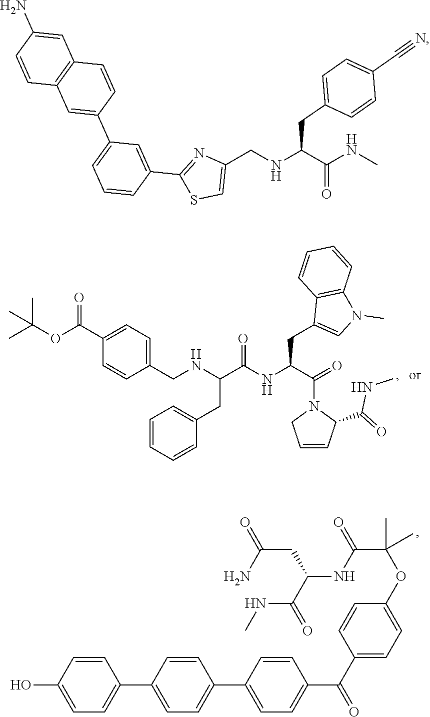 [SMILES: CNC(=O)[C@H](Cc1ccc(C#N)cc1)NCc1csc(-c2cccc(-c3ccc4cc(N)ccc4c3)c2)n1 ; CNC(=O)[C@@H]1C=CCN1C(=O)[C@H](Cc1cn(C)c2ccccc12)NC(=O)C(Cc1ccccc1)NCc1ccc(C(=O)OC(C)(C)C)cc1 ; CNC(=O)[C@H](CC(N)=O)NC(=O)C(C)(C)Oc1ccc(C(=O)c2ccc(-c3ccc(-c4ccc(O)cc4)cc3)cc2)cc1]

or a combination thereof. In some cases, the compound is

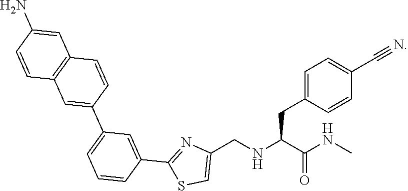 [SMILES: CNC(=O)[C@H](Cc1ccc(C#N)cc1)NCc1csc(-c2cccc(-c3ccc4cc(N)ccc4c3)c2)n1]

Also provided herein are methods of positively modulating the cannabinoid 1 receptor (CB1) comprising contacting CB1 with a positive allosteric modulator (PAM) in an amount effective to positively modulate CB1, wherein the CB1-PAM is:

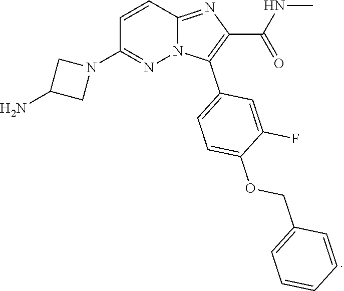 [SMILES: CNC(=O)c1nc2ccc(N3CC(N)C3)nn2c1-c1ccc(OCc2ccccc2)c(F)c1]

Further provided herein are methods for treating pain in a subject suffering therefrom comprising administering to the subject a therapeutically effective amount of a compound having a structure of

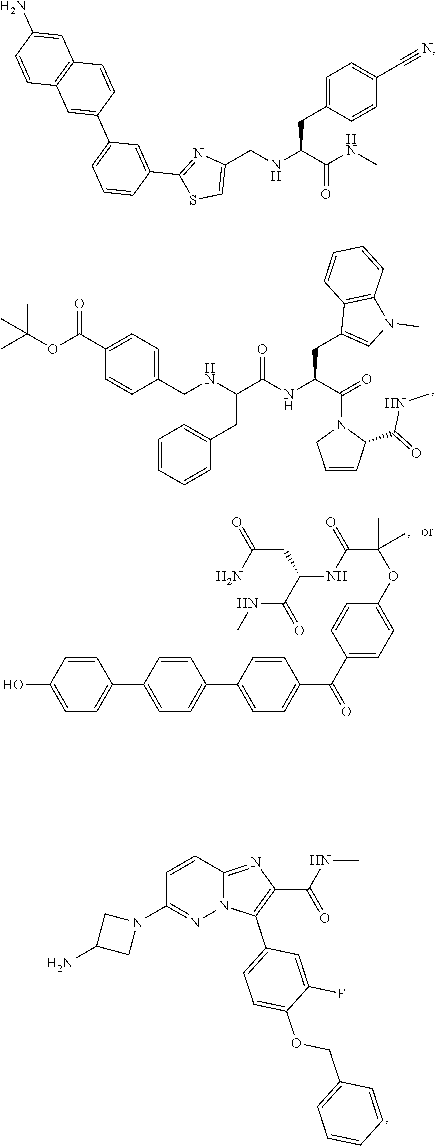 [SMILES: CNC(=O)[C@H](Cc1ccc(C#N)cc1)NCc1csc(-c2cccc(-c3ccc4cc(N)ccc4c3)c2)n1 ; CNC(=O)[C@@H]1C=CCN1C(=O)[C@H](Cc1cn(C)c2ccccc12)NC(=O)C(Cc1ccccc1)NCc1ccc(C(=O)OC(C)(C)C)cc1 ; CNC(=O)[C@H](CC(N)=O)NC(=O)C(C)(C)Oc1ccc(C(=O)c2ccc(-c3ccc(-c4ccc(O)cc4)cc3)cc2)cc1 ; CNC(=O)c1nc2ccc(N3CC(N)C3)nn2c1-c1ccc(OCc2ccccc2)c(F)c1]

or a combination thereof.

Also provided herein are methods of inhibiting μ opioid receptor (MOR), comprising contacting MOR with a compound having a structure of

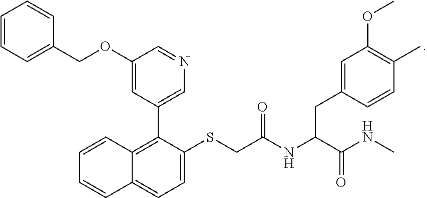 [SMILES: CNC(=O)C(Cc1ccc(C)c(OC)c1)NC(=O)CSc1ccc2ccccc2c1-c1cncc(OCc2ccccc2)c1]

Also provided herein are methods for treating opioid overdose in a subject suffering therefrom comprising administering to the subject a therapeutically effective amount of a compound having a structure of

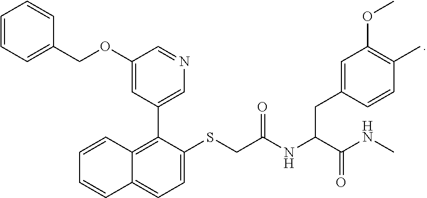 [SMILES: CNC(=O)C(Cc1ccc(C)c(OC)c1)NC(=O)CSc1ccc2ccccc2c1-c1cncc(OCc2ccccc2)c1]

In many cases, the methods for treating opioid overdose in a subject further comprises administration of a therapeutically effective amount of naloxone either before, after, or concomitantly with administration of the compound.

Further provided herein are compounds, or pharmaceutically acceptable salts thereof, wherein the compound is:

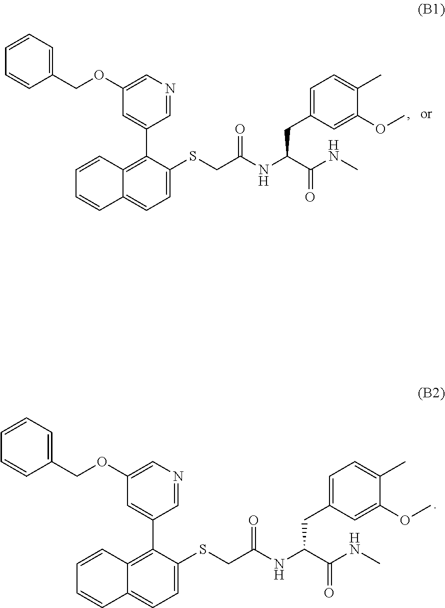 [SMILES: CNC(=O)[C@H](Cc1ccc(C)c(OC)c1)NC(=O)CSc1ccc2ccccc2c1-c1cncc(OCc2ccccc2)c1 ; CNC(=O)[C@@H](Cc1ccc(C)c(OC)c1)NC(=O)CSc1ccc2ccccc2c1-c1cncc(OCc2ccccc2)c1]

Also provided herein are methods of inhibiting μ opioid receptor (MOR), comprising contacting MOR with a compound, wherein the compound is B1 or B2.

### BRIEF DESCRIPTION OF THE DRAWINGS

FIG. 1 shows the structure of several compounds of the disclosure (Cmpds A1-A5) with their IC50 and EC50 for modulating MOR or CB1 activities.

FIG. 2 shows A) GTP turnover assay for an increasing amount of A1 and met-enkephalin; B) binding of ³H-naloxone with A1; C) GTP turnover assay for A1 alone, and with DAMGO; D) GTP turnover assay for an increasing amount of A3 and met-enkephalin; E) binding of ³H-naloxone with A3; F) GTP turnover assay for A3 alone, and with DAMGO.

FIG. 3 shows A) GTP turnover assay for an increasing amount of A4 and FUB; B) radioligand binding assay for A4 and ³H-SR141716A, compared with ZCZ; C) GTP turnover assay for A4 with FUB-bound and AEA bound CB1 receptor.

FIG. 4 shows A) antinociceptive time course experiment for morphine, morphine and naloxone, and morphine, naloxone, and various doses of compound A3; B) locomotion time course experiment for morphine, morphine and naloxone, and morphine, naloxone, and various doses of compound A3; C) respiratory time course experiment for morphine, morphine and naloxone, and morphine, naloxone, and various doses of compound A3; D) CPP results for compound A3 and naloxone, versus saline and naloxone.

FIG. 5 shows A)³H-naloxone binding for compounds A3, B1 and B2; B) GTP turnover assay for compounds A3, B1 and B2.

### DETAILED DESCRIPTION

Traditional opioids share a similar mode of action as agonists of the μ-opioid receptor (MOR). Agonists and antagonists of the MOR bind at an overlapping orthosteric site in the extracellular vestibule of the receptor and share interactions with endogenous opioid signaling peptides. Modulation of receptor activity via binding of molecules at alternate sites on the receptor (allosteric sites), rather than agonists/antagonists which bind at traditional orthosteric sites, potentially provides a series of advantages. Specifically, molecules that bind at sites distinct from the MOR orthosteric site and positively or negatively modulate its activity (PAM & NAM, respectively) have the potential to signal more specifically through the μ-opioid receptor (MOR) (rather than its κ- and δ-opioid receptor counterparts).

MOR PAMs can work synergistically with the body's endogenous opioid systems to enhance natural analgesic priorities and MOR NAMs may enhance the anti-overdose properties of naloxone. So-called ‘ceiling effects’ of allosteric modulators, dependent on the cooperativity between the allosteric site and orthosteric site, can also result in diminished propensity for MOR-induced overdose¹¹. Allosteric modulators may be able to selectively enhance the activity of certain orthosteric molecules. G-protein coupled receptors (GPCRs) are known to signal through multiple intracellular effectors, including G-proteins, G-protein coupled receptor kinases (GRKs), and the β-arrestins. Orthosteric ligands, including those for the opioid receptors⁷ and cannabinoid receptors¹², are able to activate these different signaling pathways to varying extents, a phenomenon known as bias¹¹¹. So-called biased allosteric modulators (BAMs) are able to combine the advantages of allosteric modulators above while also selectively activating or inhibiting certain desired intracellular pathways¹⁴.

Small molecules, including cannabinoids¹⁸ and the selective KOR agonist salvinorin A¹⁹, have been identified as NAMs of the MOR, though their potencies for their selective targets (CB1 and KOR) are much higher than those observed against the MOR (high μM), suggesting they are highly unlikely to display selective MOR-NAM activity in vivo. Thus, selective and potent NAMs for the MOR have remained elusive.

Additional studies have shown that ZCZ-011, a positive allosteric modulator (PAM) of CB1 enhances the antinociceptive effects without displaying psychoactive side-effects²². These properties make selective CB1 PAMs a desirable therapeutic target.

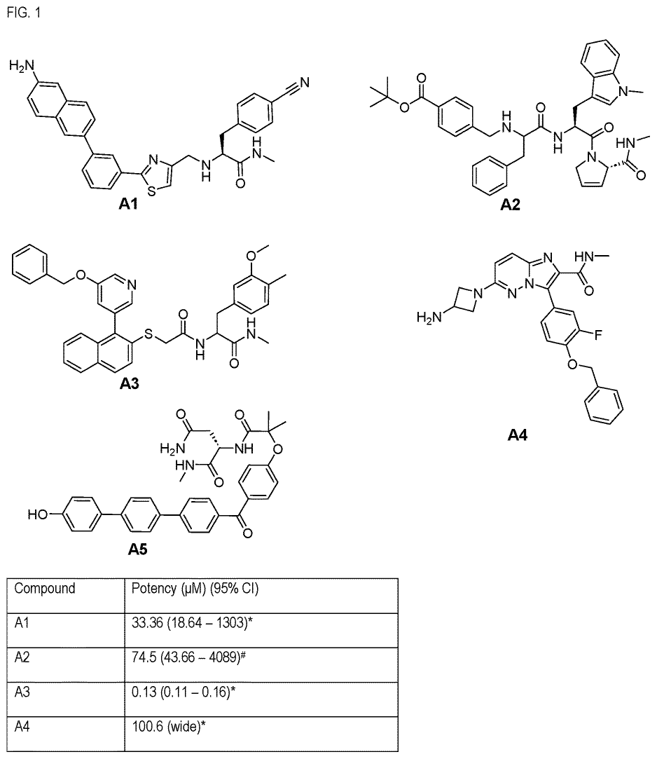

Disclosed herein are modulators of MOR and CB1, having structures as shown in FIG. 1, A1-A5.

In some cases, the compound is a MOR PAM with a structure of

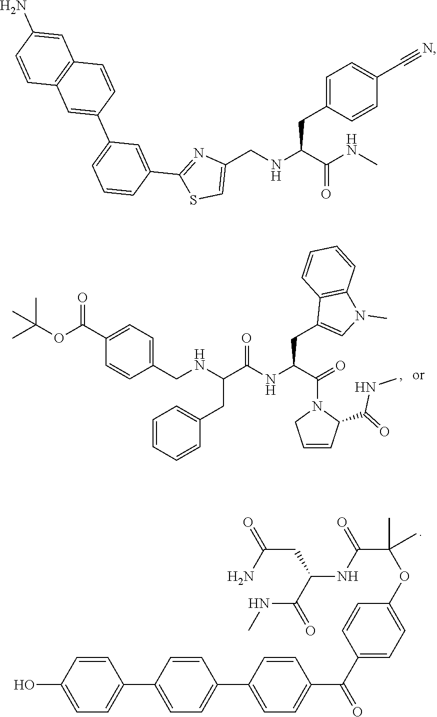 [SMILES: CNC(=O)[C@H](Cc1ccc(C#N)cc1)NCc1csc(-c2cccc(-c3ccc4cc(N)ccc4c3)c2)n1 ; CNC(=O)[C@@H]1C=CCN1C(=O)[C@H](Cc1cn(C)c2ccccc12)NC(=O)C(Cc1ccccc1)NCc1ccc(C(=O)OC(C)(C)C)cc1 ; CNC(=O)[C@H](CC(N)=O)NC(=O)C(C)(C)Oc1ccc(C(=O)c2ccc(-c3ccc(-c4ccc(O)cc4)cc3)cc2)cc1]

In some cases, the compound is a CB1PAM with a structure of

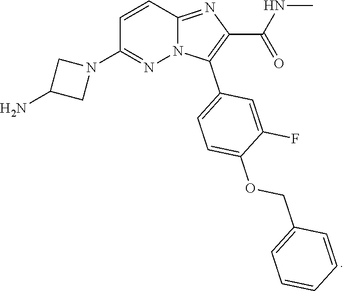 [SMILES: CNC(=O)c1nc2ccc(N3CC(N)C3)nn2c1-c1ccc(OCc2ccccc2)c(F)c1]

In some cases, the compound is a MOR NAM with a structure of

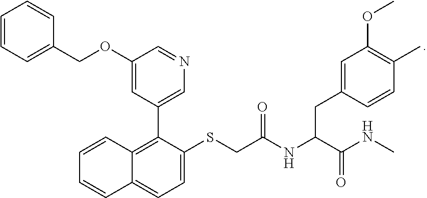 [SMILES: CNC(=O)C(Cc1ccc(C)c(OC)c1)NC(=O)CSc1ccc2ccccc2c1-c1cncc(OCc2ccccc2)c1]

Also disclosed herein are inhibitors of MOR having a structure of:

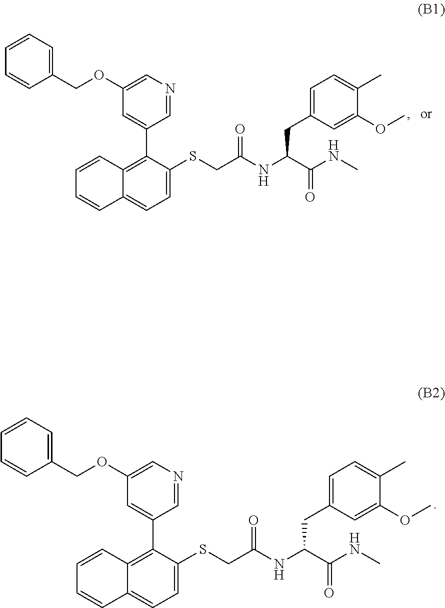 [SMILES: CNC(=O)[C@H](Cc1ccc(C)c(OC)c1)NC(=O)CSc1ccc2ccccc2c1-c1cncc(OCc2ccccc2)c1 ; CNC(=O)[C@@H](Cc1ccc(C)c(OC)c1)NC(=O)CSc1ccc2ccccc2c1-c1cncc(OCc2ccccc2)c1]

The MOR or CB1 modulators disclosed herein can be used in methods of modulating MOR or CB1 in a cell. In these methods, a cell is contacted with a disclosed MOR or CB1 modulator, or pharmaceutical composition thereof, in an amount effective to modulate MOR or CB1. In various cases, the contacting includes administering the compound or pharmaceutical composition to a subject.

### Pharmaceutically Acceptable Salts

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxyl group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Targets for Treatment

The compounds disclosed herein can be used in methods for positively modulating μ opioid receptor (MOR PAM), positively modulating cannabinoid 1 receptor (CB1PAM), or negatively modulating μ opioid receptor (MOR NAM).

The MOR or CB1PAM compounds disclosed herein can be used in methods of treating pain in a subject. In some cases, the compound used in methods of treating pain is a MOR PAM. In some cases, the compound used in methods of treating pain is a CB1PAM.

The MOR NAM compounds of the disclosure can be used in methods of treating opioid overdose in a subject. In various cases, naloxone can optionally be administered either before, after, or concomitantly with administration of the MOR NAM compound.

### Pharmaceutical Formulations

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g. orally, topically, rectally, parenterally, (for example, subcutaneous injections, intravenous, intramuscular, intrasternal, and intrathecal injection or infusion techniques), or as a buccal, inhalation, or nasal spray. The administration can be to provide a systemic effect (e.g. eneteral or parenteral). All methods that can be used by those skilled in the art to administer a pharmaceutically active agent are contemplated. In some cases, the disclosed formulations can be administered orally or topically.

Suitable oral compositions or formulations in accordance with the disclosure include without limitation tablets, troches, lozenges, aqueous or oily suspensions, dispersible powders or granules, emulsion, hard or soft capsules, syrups or elixirs. Compositions or formulations suitable for oral use may be prepared according to any method known to the art for the manufacture of pharmaceutical compositions.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions and formulations described herein may also be administered topically or transdermally, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract, e.g., can be effected in a rectal suppository formulation or in a suitable enema formulation. Dosage forms for topical or transdermal administration of a compound described herein include ointments, pastes, creams, lotions, gels, powders, solutions, sprays, inhalants, suppositories, or patches.

For topical applications, the pharmaceutical compositions may be formulated in a suitable ointment, cream, lotion, or gel, containing the active component suspended or dissolved in one or more carriers, and any needed preservatives or buffers as may be required. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EMBODIMENTS OF THE DISCLOSURE

1. A method of positively modulating μ opioid receptor (MOR) comprising contacting MOR with a compound have a structure of

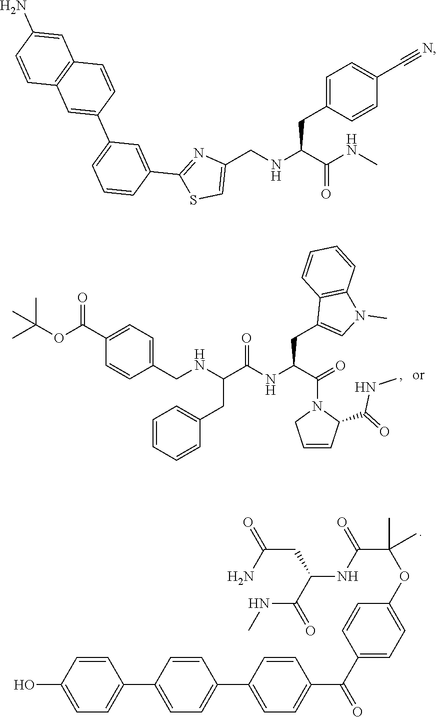 [SMILES: CNC(=O)[C@H](Cc1ccc(C#N)cc1)NCc1csc(-c2cccc(-c3ccc4cc(N)ccc4c3)c2)n1 ; CNC(=O)[C@@H]1C=CCN1C(=O)[C@H](Cc1cn(C)c2ccccc12)NC(=O)C(Cc1ccccc1)NCc1ccc(C(=O)OC(C)(C)C)cc1 ; CNC(=O)[C@H](CC(N)=O)NC(=O)C(C)(C)Oc1ccc(C(=O)c2ccc(-c3ccc(-c4ccc(O)cc4)cc3)cc2)cc1]

or a combination thereof, in an amount sufficient to positively modulate MOR.

2. The method of embodiment 1, wherein the compound is

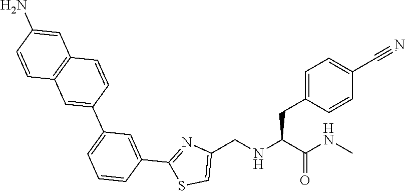 [SMILES: CNC(=O)[C@H](Cc1ccc(C#N)cc1)NCc1csc(-c2cccc(-c3ccc4cc(N)ccc4c3)c2)n1]

3. A method of positively modulating cannabinoid 1 receptor (CB1) comprising contacting CB1 with a compound having a structure

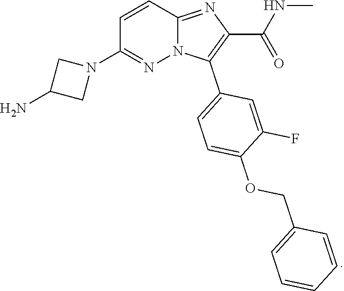 [SMILES: CNC(=O)c1nc2ccc(N3CC(N)C3)nn2c1-c1ccc(OCc2ccccc2)c(F)c1]

in an amount sufficient to positively modulate CB1.

4. A method for treating pain in a subject suffering therefrom comprising:
administering to the subject a therapeutically effective amount of a compound having a structure of

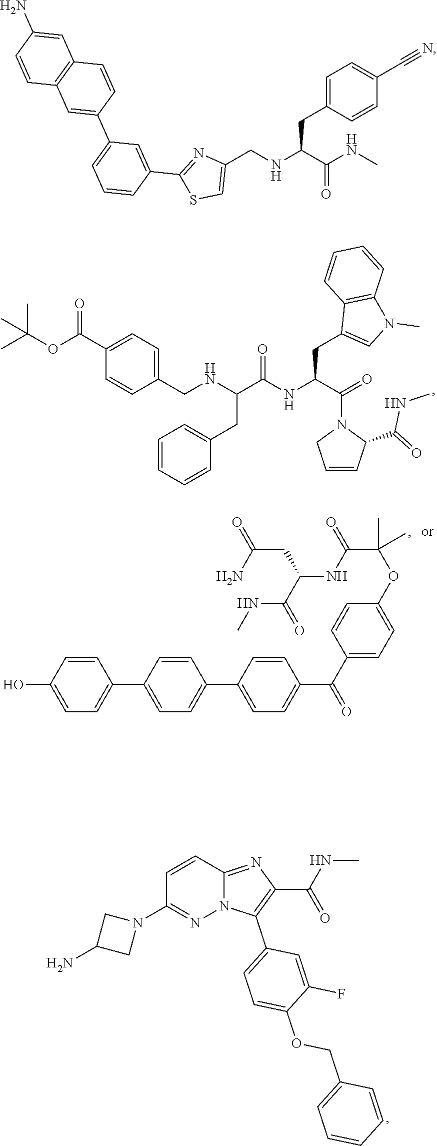 [SMILES: CNC(=O)[C@H](Cc1ccc(C#N)cc1)NCc1csc(-c2cccc(-c3ccc4cc(N)ccc4c3)c2)n1 ; CNC(=O)[C@@H]1C=CCN1C(=O)[C@H](Cc1cn(C)c2ccccc12)NC(=O)C(Cc1ccccc1)NCc1ccc(C(=O)OC(C)(C)C)cc1 ; CNC(=O)[C@H](CC(N)=O)NC(=O)C(C)(C)Oc1ccc(C(=O)c2ccc(-c3ccc(-c4ccc(O)cc4)cc3)cc2)cc1 ; CNC(=O)c1nc2ccc(N3CC(N)C3)nn2c1-c1ccc(OCc2ccccc2)c(F)c1]

or a combination thereof.

5. The method of embodiment 4, wherein the compound is

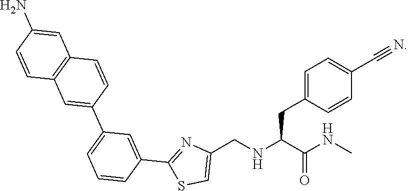 [SMILES: CNC(=O)[C@H](Cc1ccc(C#N)cc1)NCc1csc(-c2cccc(-c3ccc4cc(N)ccc4c3)c2)n1]

6. The method of embodiment 4, wherein the compound is

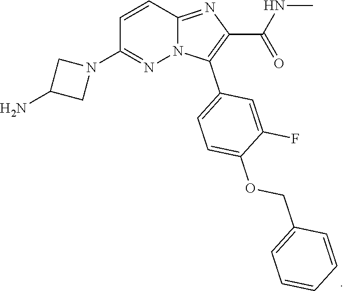 [SMILES: CNC(=O)c1nc2ccc(N3CC(N)C3)nn2c1-c1ccc(OCc2ccccc2)c(F)c1]

7. A method of inhibiting μ opioid receptor (MOR), comprising contacting MOR with a compound having a structure of

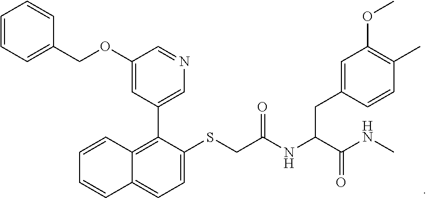 [SMILES: CNC(=O)C(Cc1ccc(C)c(OC)c1)NC(=O)CSc1ccc2ccccc2c1-c1cncc(OCc2ccccc2)c1]

8. A method for treating opioid overdose in a subject suffering therefrom comprising administering to the subject a therapeutically effective amount of a compound having a structure of

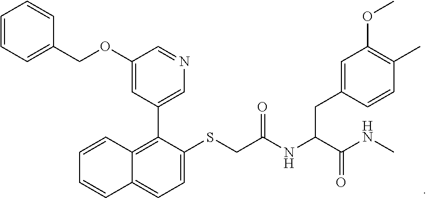 [SMILES: CNC(=O)C(Cc1ccc(C)c(OC)c1)NC(=O)CSc1ccc2ccccc2c1-c1cncc(OCc2ccccc2)c1]

9. The method of embodiment 8, further comprising administration of a therapeutically effective amount of naloxone either before, after, or concomitantly with administration of the compound.

10. A compound, or pharmaceutically acceptable salt thereof, wherein the compound is

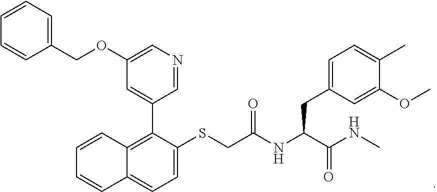 [SMILES: CNC(=O)[C@H](Cc1ccc(C)c(OC)c1)NC(=O)CSc1ccc2ccccc2c1-c1cncc(OCc2ccccc2)c1]

11. The compound, or pharmaceutically acceptable salt thereof, wherein the compound is

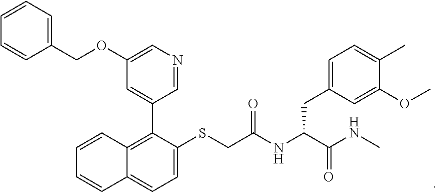 [SMILES: CNC(=O)[C@@H](Cc1ccc(C)c(OC)c1)NC(=O)CSc1ccc2ccccc2c1-c1cncc(OCc2ccccc2)c1]

12. A method for inhibiting μ opioid receptor (MOR), comprising contacting MOR with the compound of claim 10 or 11 in an amount sufficient to inhibit MOR.

### EXAMPLES

### Experimental Procedures

### Chemical Materials

Reagents were purchased from Sigma-Aldrich Chemicals, Ambeed, Chemscene, Chemimpex and used as-is. All reactions were performed under argon atmosphere unless otherwise specified. While performing synthesis, reaction mixtures were purified by silica gel flash chromatography on E. Merck 230-400 mesh silica gel 60 using a Teledyne ISCO CombiFlash Rf instrument with UV detection at 280 and 254 nm. RediSep Rf silica gel normal phase columns were used with a gradient range of 0-80% EtOAc in Hexane. Final clean (purity ≥95%, LC-MS Agilent 1100 Series LC/MSD) compounds were used for the study. NMR spectra were collected using Varian 500 MHz NMR instrument and collected via the Bruker Topspin Software (Bruker Topspin 3.5 pl 6). Chemical shifts are reported in parts per million (ppm) relative to residual solvent peaks at the nearest 0.01 for proton and 0.1 for carbon (CDCl₃ ¹H: 7.26, ¹³C: 77.1). Peak multiplicity is reported as the NMR spectra were processed with MestreNova software 14.2.0, namely s—singlet, d—doublet, t—triplet, q—quartet, m—multiplet for examples. Coupling constant (J) values are expressed in Hz. Mass spectra were obtained using an Agilent 1100 Series LC/MSD by electrospray (ESI) ionization with a gradient elution program (Ascentis Express Peptide C18 column, acetonitrile/water 5/95/95/5, 5 minutes, 0.05% formic acid) and UV detection (214 nM/254 nM). High resolution mass spectra were obtained using a Bruker 10 T APEX-Qe FTICR-MS and the accurate masses are reported for the molecular ion [M+H]⁺.

### Purification of μ-Opioid Receptor and Variants

The mouse μ-opioid receptor was grown and purified as previously described²⁹. Mouse μ-opioid receptor (MOR) with an N-terminal FLAG and C-terminal hexa-histidine tag was expressed as previously described²⁹ using the baculovirus method in Spodoptera frugiperda (Sf9) cells. Naloxone was added to 10 μM final concentration upon infection and cells were collected 48 hours post-infection and stored at −80° C. for later purification. MOR was extracted from membranes with 0.8% n-dodecyl-ß-D-malopyranoside (DDM; Anatrace), 0.08% cholesterol hemisuccinate (CHS), and 0.3% 3-((3-cholamidopropyl) dimethylammonio)-1-propanesulfonate (CHAPS; Anatrace) in 20 mM HEPES pH 7.5, 500 mM sodium chloride (NaCl), 30% glycerol, 5 mM imidazole, 10 μM naloxone, and the protease inhibitors benzamidine and leupeptin, along with benzonase (Sigma-Aldrich) to degrade cellular DNA. Cells were dounced 30 times on ice and the membranes were allowed to solubilize in the detergent for 2 hours with stirring at 4° C., followed by centrifugation for 40 minutes at 14k rpm to pellet cell debris. The supernatant was applied to nickel-chelating sepharose resin and bound to resin with end-over-end shaking for 2 hours at 4° C. The resin was then batch washed 4 times followed by washing with 10 column volumes on-column with nickel wash buffer composed of 20 mM HEPES pH 7.4, 500 mM NaCl, 0.1% DDM, 0.01% CHS, 0.03% CHAPS, 5 mM imidazole, 10 μM naloxone, and protease inhibitors leupeptin and benzamidine. The nickel-pure MOR was then eluted in the same buffer with 250 mM imidazole. The nickel elution was initially exchanged to lauryl maltose neopentyl glycol (L-MNG; Anatrace) detergent by incubating with 0.5% L-MNG, 0.17% glycol-diosgenin (GDN; Anatrace) and 0.067% CHS overnight at 4° C. 2 mM calcium chloride (CaCl₂) was then added and subsequently applied to M1 anti-FLAG immunoaffinity resin. The M1-bound receptor was first washed with 20 mM HEPES pH 7.4, 500 mM NaCl, 0.1% MNG, 0.033% GDN, 0.0133% CHS, 2 mM CaCl2, and 10 μM naloxone. The protein was then washed with 10 column volumes of 20 mM HEPES pH 7.4, 100 mM NaCl, 0.005% MNG, 0.0017% GDN, 0.00067% CHS and 2 mM CaCl2 followed by elution with 20 mM HEPES pH 7.4, 100 mM NaCl, 0.003% MNG, 0.001% GDN, 0.0004% CHS, 5 mM ethylenediaminetetraacetic acid (EDTA) and FLAG peptide. Multimers and dimers of the receptor were removed with size exclusion chromatography on an S200 10/300 Increase gel filtration column (GE Healthcare) equilibrated with 20 mM HEPES pH 7.4, 100 mM NaCl, 0.003% MNG, 0.001% GDN, 0.0004% CHS. Pure, monomeric apo MOR was spin concentrated to −150 μM, flash frozen in liquid nitrogen and stored at −80° C. until further use.

### Purification of Cannabinoid Receptor 1

Purification of human full-length CB1 with an N-terminal FLAG and C-terminal hexahistidine tags was performed as previously described using the baculovirus method (Expression Systems).²² Briefly, CB1 was extracted from insect cell membranes with 1% L-MNG and 0.1% CHS and purified by nickel-chelating Sepharose chromatography. Crude, nickel-pure CB1 was applied to M1 anti-FLAG immunoaffinity resin and washed with decreasing amounts of inverse agonist SR. The receptor was eluted from M1 resin with a buffer consisting of 20 mM HEPES pH 7.5, 150 mM NaCl, 0.05% L-MNG, 0.005% CHS, FLAG peptides and 5 mM EDTA. The FLAG-pure CB1 was further purified by size exclusion chromatography on an S200 10/300 Increase gel filtration column (GE Healthcare) equilibrated with 20 mM HEPES pH 7.5, 150 mM NaCl, 0.05% L-MNG and 0.005% CHS. Purified, monomeric CB1 was concentrated to ~500 μM and flash frozen and stored at −80° C. until further use.

### Expression & Purification of Heterotrimeric G-Proteins

Heterotrimeric Gᵢ was expressed in Trichoplusia ni (T. ni) with the BestBac method (Expression Systems) and purified as previously described.^{2, 29} Briefly, T. ni cells were infected with one virus encoding the wild-type human Gαᵢ subunit and another encoding the wild-type human β₁γ₂ subunits with a histidine tag inserted at the N-terminus of the β subunit. Cells were harvested 48 hours post infection and lysed with hypotonic buffer. Heterotrimeric Gαβ₁γ₂ proteins were extracted in a buffer containing 1% sodium cholate and 0.05% DDM. The heterotrimer was purified using nickel-chelating sepharose chromatography while removing cholate. Human rhinovirus 3C protease (3C protease) was added to cleave off the histidine tag overnight at 4° C. on-column. The flow through was collected and dephosphorylated with lambda protein phosphatase (NEB), calf intestinal phosphatase (NEB) and Antarctic phosphatase (NEB) in the presence of 1 mM manganese chloride (MnCl₂). The heterotrimer was further purified by ion exchange chromatography on a MonoQ 10/100 GL column (GE Healthcare) in 20 mM HEPES pH 7.5, 1 mM MgCl₂, 0.05% DDM, 100 μM TCEP, and 20 μM GDP and eluted with a linear NaCl gradient from 50 to 500 mM. The purified heterotrimer was collected and dialyzed into 20 mM HEPES pH 7.5, 100 mM NaCl, 0.02% DDM, 100 μM TCEP and 20 μM GDP overnight at 4° C., followed by concentration to <250 μM, addition of 20% glycerol and flash-freezing in liquid nitrogen and storage at −80° C. until further use.

Purification of scFv16

scFv16 was grown and purified as previously described²⁹. Briefly, scFv with a hexahistidine tag was secreted from T. ni Hi5 cells and purified by nickel-chelating sepharose resin chromatography. The protein was eluted from nickel resin with 20 mM HEPES pH 7.4, 500 mM NaCl, and 250 mM imidazole and incubated with 3C protease during dialysis overnight into 20 mM HEPES pH 7.4 and 100 mM NaCl in order to cleave off the hexahistidine tag. The protein was further purified by reverse nickel chromatography after cleavage, followed by size exclusion with a S200 10/300 Increase column into 20 mM HEPES pH 7.4 and 100 mM NaCl. Monomeric scFv16 fractions were pooled, concentrated, and flash-frozen in liquid nitrogen for later experiments.

Purification of Human μ-Opioid Receptor from Expi293 Cells

The gene for the full-length human μ-opioid receptor was cloned into a vector for inducible expression in Expi293F cells (Thermo Fisher) with N-terminal HA signal peptide and FLAG tags and a C-terminal hexahistidine tag. This construct was transfected into Expi293F cells which were constitutively expressing the tetracycline repressor (Thermo Fisher) using the Expifectamine transfection kit (Thermo Fisher), according to the instructions by the manufacturer. Then, 2 days after transfection, receptor expression was induced with doxycycline (4 μg/mL and 5 mM sodium butyrate) in the presence of 10 μM naloxone. Next, −30 hours after induction, pellets were collected and frozen at −80° C. for later protein purification. Cells were dounced and membranes were solubilized with 20 mM HEPES pH 7.5, 100 mM NaCl, 20% glycerol, 1% L-MNG, 0.1% CHS, 10 μM naloxone, protease inhibitors leupeptin and benzamidine, and benzonase, followed by purification on anti-FLAG immunoaffinity resin as above. Multimers and dimers of the receptor were removed with size exclusion chromatography on an S200 10/300 Increase gel filtration column (GE Healthcare) equilibrated with 20 mM HEPES pH 7.4, 100 mM NaCl, 0.003% MNG, 0.001% GDN, 0.0004% CHS. Pure, monomeric apo MOR was spin concentrated to ~150 μM, flash frozen in liquid nitrogen and stored at −80° C. until further use.

### Preparation of Samples for DEL Selections

### MOR/G/Met-Enkephalin Complex Formation

MOR/Gᵢ complex was formed in a similar manner to that described previously^{29, 31}. Briefly, excess met-enkephalin peptide [MedChemExpress] was added to purified MOR (above) and incubated at room temperature for 1 hour. Concurrently, 1% L-MNG/0.1% CHS was added to Gᵢ purified in DDM to exchange detergent on ice for 1 hour. The two reactions were mixed for a final molar ratio of 1:1.5 MOR:Gᵢ and incubated at room temperature for 1 hour. Apyrase was added and the reaction was further incubated on ice for 2 hours. 2 mM CaCl₂ was added to the reaction before adding to M1 anti-FLAG resin for 20 minutes. The column was washed with 20 mM HEPES pH 7.4, 100 mM NaCl, 0.003% MNG, 0.001% GDN, 0.0004% CHS, 5 μM met-enkephalin, and 2 mM CaCl₂, followed by elution in the same buffer with 5 mM EDTA replacing the CaCl₂ and FLAG peptides. After removing excess unbound Gᵢ heterotrimer, excess un-coupled MOR was removed by size exclusion chromatography with a S200 10/300 Increase column into 20 mM HEPES pH 7.4 and 100 mM NaCl, 0.003% MNG, 0.001% GDN, 0.0004% CHS, 100 μM TCEP and 1 μM met-enkephalin. MOR/Gᵢ/met-enkephalin complex was spin concentrated to ~25 μM and flash-frozen in liquid nitrogen after adding 15% glycerol.

### MOR/Naloxone Formation

Purified mouse MOR (above) was incubated with excess naloxone for 1 hour at room temperature before selection experiments in 20 mM HEPES pH 7.4, 100 mM NaCl, 0.003% MNG, 0.001% GDN, 0.0004% CHS.

### CB1/G/MDMB-Fubinaca Complex Formation

CB1/Gᵢ complex was formed in a similar manner to that described previously²⁵.

### DEL Selection

### Target Preparation

The three samples produced above (MOR/Gi/met-enkephalin, MOR/naloxone, and CB1/Gi/MDMB-fubinaca) were diluted to 2 μM final concentration in W1: 20 mM HEPES pH 7.4, 150 mM NaCl, 100 μM TCEP, 0.1% hsDNA (Thermo Fisher Scientific), 0.02% MNG, 0.002% CHS and 20 μM ligand (met-enkephalin, naloxone, and MDMB-fubinaca, respectively). A fourth blank sample (no protein control) was made with the same buffer in the absence of ligand. 300 μL of HisPur™ Ni-NTA magnetic beads (Thermo Fisher Scientific) as slurry were washed with water followed by splitting into 4 batches and washing with the 4 respective buffers made above. Washing with buffer was repeated 2 additional times. 300 μL of 2 μM targets (above) and 300 μL buffer without agonist was incubated with the respective magnetic Ni-NTA resin for 30 minutes with mixing to bind targets to resin. Each of the 4 samples was then split into 3 100 μL aliquots corresponding to each of the 3 rounds of selection.

First round of selection: Any unbound target was removed from the 4 100 μL targets by washing with 200 μL respective wash buffer above. The 4 DEL samples (G1, G2, G3, G4) provided by WuXi (10 μL) were resuspended with 90 μL of the respective wash buffers above for the 4 conditions and then applied to the 4 targets bound to magnetic resin and incubated with shaking at room temperature for 1.5 hours to bind to the target(s). Reactions were spun briefly to settle the resin, followed by washing all with 200 μL respective buffer above. The 4 selections were then washed again with 200 μL of the same buffers with lower detergent concentrations (0.001% MNG, 0.0001% CHS) and lower ligand concentrations (500 nM) (W2). The 4 selections were then washed with 200 μL of the same respective buffers in the absence of detergent and even further lower ligand concentration (100 nM) (W3) to minimize detergent and ligand carry-over between rounds. The resin with target (and bound library molecules) was then resuspended in 100 μL of the respective last wash buffer and heated at 95° C. for 10 minutes to elute bound library components but leave receptor bound to resin. The heat denatured mixture was spun down and the supernatant removed from the resin. 10 μL of each of the 4 selections was saved for later analysis, and 90 μL was reserved for the next round of selection.

Second round of selection: Another 100 μL aliquot of target-bound magnetic resin was washed with 200 μL respective W1 buffer to remove unbound target. The 4 samples of resin were then resuspended in 90 μL respective W1, to which the 90 μL of reserved Round 1 DEL selection was added and incubated with shaking at room temperature for 1.5 hours. Reactions were spun briefly to settle the resin, followed by washing with 200 μL respective W1 buffer. The 4 selections were then washed again with 200 μL of respective W2 buffer, followed by 200 μL of respective W3 buffer. The resin with target (and bound library molecules) was then resuspended in 100 μL respective W3 and heated at 95° C. for 10 minutes to elute bound library components but leave receptor bound to resin. The heat denatured mixture was spun down and the supernatant removed from the resin. 50 μL of each of the 4 selections was saved for later analysis, and 50 μL was reserved for the next round of selection.

Third round of selection: Another 100 μL aliquot of target-bound magnetic resin was washed with 200 μL respective W1 buffer to remove unbound target. The 4 samples of resin were then resuspended in 50 μL respective W1, to which the 50 μL of reserved Round 2 DEL selection was added and incubated with shaking at room temperature for 1.5 hours. Reactions were spun briefly to settle the resin, followed by washing with 200 μL respective W1 buffer. The 4 selections were then washed again with 200 μL of respective W2 buffer, followed by 200 μL of respective W3 buffer. The resin with target (and bound library molecules) was then resuspended in 100 μL respective W3 and heated at 95° C. for 10 minutes to elute bound library components but leave receptor bound to resin. The heat denatured mixture was spun down and the supernatant removed from the resin. All of the final (third) round of selection supernatant for all 4 conditions was saved for subsequent sequencing and enrichment analysis.

### Preparation of μ-Opioid Receptor Membranes

Cell membranes containing the mouse MOR described above were generated by infecting Sf9 cells in an identical manner to that described above for protein purification. Sf9 cells expressing MOR were resuspended in cold lysis buffer composed of 10 mM HEPES pH 7.4, 10 mM MgCl₂, and 20 mM potassium chloride (KCl) with the protease inhibitors leupeptin, benzamidine, and cOmplete™ EDTA-free protease inhibitor cocktail tablets (Sigma-Aldrich). The lysed cells were spun at 45k rpm for 45 minutes to pellet membranes. The supernatant was removed and membrane pellets were resuspended in cold lysis buffer followed by douncing ~30 times on ice. The dounced membranes were spun again at 45k rpm for 45 minutes. The pellets were resuspended in cold lysis buffer again with the addition of benzonase and dounced a further −30 times, followed by a further spin at 45k rpm for 45 minutes. Membranes were resuspended in the same lysis buffer above in the presence of 1 M NaCl and dounced a further ~30 times and pelleted. Finally, membranes were washed with the original lysis buffer, dounced, and pelleted. Final washed membranes were resuspended to 2 g original pellet mass per 1 mL in lysis buffer; the resulting MOR-containing membranes were flash frozen for later radioligand binding experiments.

### Radioligand Binding Experiments

### Membrane Binding

MOR-containing membranes prepared above were diluted 1:1000 in 20 mM HEPES pH 7.4, 100 mM NaCl, and 0.05% bovine serum albumin (BSA). For saturation binding experiments, membranes were incubated with a serial dilution of ³H-naloxone (50.3 Ci/mmol; Perkin Elmer) and allosteric modulators at different constant concentrations for 1.5 hours at room temperature with shaking. For “competition” binding experiments, membranes were incubated with 2 nM ³H-naloxone and serially diluted allosteric modulators for 1.5 hours at room temperature with shaking. Following incubation, membranes were rapidly bound to double thick 90×120 mm glass fibre Printed Filtermat B filters (Perkin Elmer) and washed with cold binding buffer (20 mM HEPES pH 7.4, 100 mM NaCl) using a MicroBeta Filtermat-96 cell harvester (Perkin Elmer). ³H-naloxone bound membranes on Filtermats were measured with a MicroBeta counter (Perkin Elmer) after addition of MultiLex B/HS melt-on scintillator sheets (Perkin Elmer) and data values were plotted as total counts per minute normalized to the highest and lowest average points.

CB1-containing membranes prepared in a similar manner to the μOR-containing membranes prepared above were diluted 1:2000 in 50 mM HEPES pH 7.4, 5 mM EDTA, 5 mM MgCl₂, and 1% bovine serum albumin (BSA). 96-well plates precoated with blocking buffer (20 mM HEPES (pH 7.4), 100 mM NaCl, and 0.05% BSA) were used as reaction plates. For “competition” binding experiments, membranes were incubated with 6 nM ³H-SR141716a (50.9 Ci/mmol; Perkin Elmer) and serially diluted allosteric modulators for 1 hour at room temperature with shaking. Following incubation, membranes were rapidly bound to double thick 90×120 mm glass fibre Printed Filtermat B filters (Perkin Elmer) presoaked in 0.33% (v/v) polyethylenimine and washed with cold binding buffer (20 mM HEPES pH 7.4, 100 mM NaCl) using a MicroBeta Filtermat-96 cell harvester (Perkin Elmer). ³H-SR141716a bound membranes on Filtermats were measured with a MicroBeta counter (Perkin Elmer) after addition of MultiLex B/HS melt-on scintillator sheets (Perkin Elmer) and data values were plotted after normalizing total counts per minute to the highest and lowest values.

### GTP Turnover Assay

The GTP turnover assay was performed using a modified version of the GTPase-GLO™ assay (Promega) as described previously^{26, 27}. Purified MOR was diluted to 1 μM in 20 mM HEPES pH 7.4, 100 mM NaCl, 0.01% L-MNG, 0.001% CHS, and 20 μM guanosine-5′-triphosphate (GTP) in the presence of various orthosteric (20 μM met-enkephalin, 20 μM naloxone, 20 μM MP, 20 μM H-Tyr-D-Ala-Gly-N(Me)Phe-Gly-OH [DAMGO], 20 μM BU72) and allosteric (serially diluted or at excess concentrations of 20 μM for A3 or 100 μM for A1) ligands and incubated for 1.5 hours at room temperature. Concurrently, Gᵢ purified in DDM was exchanged by incubating with 1% L-MNG and 0.1% CHS for 1 hour on ice. The exchanged Gᵢ was then diluted to 1 μM in 20 mM HEPES pH 7.4, 100 mM NaCl, 0.01% L-MNG, 0.001% CHS, 20 μM guanosine-5′-diphosphate (GDP), 200 μM TCEP, and 20 mM MgCl₂. Equal volumes of receptor solutions and G solution were mixed and incubated at room temperature for 60 minutes (agonist-bound receptor experiments) or 90 minutes (apo receptor experiments) with gentle shaking. Controls include mixing equal volumes of both buffers (total initial GTP) and equal volumes of 1 μM G solution and receptor buffer (intrinsic G protein turnover). Equal volume of GTPase-Glo reagent supplemented with 10 μM adenosine 5′-diphosphate (ADP) in 20 mM HEPES pH 7.4, 100 mM NaCl, 0.01% MNG and 0.001% CHS was added and incubated with gentle shaking for 30 minutes, followed by addition of further equal volume of detection reagent. After brief (10 minute) incubation, luminescence was measured with a MicroBeta counter.

### TRUPATH Assay

The allosteric activity for the highly enriched MOR NAM (compound A3) was tested in combination with various orthosteric agonists (morphine, fentanyl, and met-enkephalin) or antagonist (naloxone). TRUPATH assays were used to determine the negative allosteric effect of compound A3 on the coupling of the MOR and heterotrimeric G proteins.³⁹ HEK293T cells were co-transfected in a 5:1:5:5 ratio of MOR, individual Ga-RLuc8 (Gᵢ₁ or Gᵢ₃), Gβ₃, and GFP2-Gγ₈ construct, while a combination of a 5:1:5:5 ratio of MOR, individual Gα-RLuc8 (Gᵢ₂, G_{oA}, or G_{oB}), Gβ₃, and GFP2-Gγ₈ construct and a 5:1:5:5 ratio of OR, individual Gα-RLuc8 (G_{z}), Gβ₃, and GFP2-Gγ₁ construct was used in the presence of transfection reagent, Transit 2020. The next day, the transfected cells were plated and incubated overnight into poly-L-lysine coated 96-well white clear bottom cell culture plates at a density of 50,000 cells per 200 μl per well using Dulbecco's modified Eagle's medium supplemented with 1% dialyzed fetal bovine serum. On the day after, cells were washed with 60 μL of a drug buffer (1×HBSS and 20 mM HEPES, pH 7.4) per well after aspirating the cells media.

To measure the allosteric activity of compound A3 with individual orthosteric agonists, 60 μL of 7.5 μM RLuc substrate coelenterazine 400a (Nanolight) was added per well, followed by the addition of 30 μL per well of 3× final concentrations (0 and 90 μM) of compound A3 that was prepared in a drug buffer supplemented with 0.3% BSA and incubated for 20 min in the dark at room temperature. Finally, 30 μL of 4× final various concentrations of morphine, fentanyl, or met-enkephalin were prepared in a drug buffer containing 0.3% BSA then added and incubated for 5 min.

To measure the allosteric activity of compound A3 with naloxone, 30 μL of 2× final various concentrations of naloxone was added per well, followed by the addition of 30 μL per well of 2× final concentrations (0 and 90 μM) of compound A3 that was prepared in a drug buffer supplemented with 0.3% BSA and incubated for 20 min.

Afterward, 60 μL of 7.5 μM RLuc substrate coelenterazine 400a (Nanolight) was loaded per well and incubated in the dark at room temperature. After 5 min, 30 μL of 5× final concentration of DAMGO's EC₅₀ prepared in a drug buffer that contained 0.3% BSA was loaded per well and incubated in the dark at room temperature for an additional 5 min. Subsequently, Mithras LB940 multimode microplate reader was used to measure the BRET ratios for Ga protein activation by quantifying the ratio of the GFP2 emission to RLuc8 emission for 1 s per well at 510 nm and 395 nm, respectively. GraphPad Prism 9 software was used to determine the potency and efficacy of the examined orthosteric agonists and the antagonist by plotting their different used concentrations against the normalized BRET ratios that were normalized by defining 0% as basal and 100% as maximum values at 0 μM compound A3.

### In-Vivo Experiments

All experiments used male C57BL/6J mice (Jackson Laboratories Bar Harbor) (20-35 g) at 5 per cage with a 12-hour light/dark cycle (i.e. lights off at 7:00 PM and on at 7:00 AM) with ad libitum access to food and water. Mice were kept at a constant temperature (20-24° C.) and relative humidity (40-50%). Mice were used in pharmacokinetics⁴⁴, warm-water tail withdrawal^{41, 45}, locomotor and respiration (via comprehensive lab animal monitoring system, CLAMS)^{70, 71}, conditioned place preference^{41, 46}, and naloxone-precipitated opioid withdrawal assays⁴³.

### Conditioned Place Preference (CPP) Assay

Groups of mice were place conditioned using a counterbalanced design in a three-chamber place conditioning system (San Diego Instruments) as described previously^{40, 41}. The amount of time individual mice spent in each compartment both before and after place conditioning was measured over a 30-min testing period, where animals freely explored the three compartments. Following determination of initial preference, mice were administered 0.9% saline, and half the sample consistently confined in a randomly assigned outer chamber for 40 min. Four hours later, mice were pretreated subcutaneously for 30 min with either vehicle or compound A3 (100 mg/kg), followed by all mice receiving low-dose naloxone (0.1 mg/kg, s.c.). Fifteen minutes later, mice were administered morphine (10 mg/kg, i.p.) and place conditioned for 40 minutes in the opposite outer chamber. Mice were place conditioned in this manner for 2 days, with a final place preference performed on the fourth day. Data are plotted as the difference in time spent in the eventual morphine-paired chamber versus the vehicle-paired compartment.

### Naloxone-Precipitated Opioid Withdrawal Assay

Mice were made physically dependent on morphine using previously established methods^{42, 43}. Briefly, mice were placed into four groups (n=10-11 per group) and were given either saline (group 1) or escalating doses of morphine (10-75 mg/kg, groups 2-4) twice daily for four days. A final dose of saline (for group 1) or morphine (25 mg/kg) was given on day 5 to assess antinociceptive tolerance. Two hours later, precipitated withdrawal symptoms were assessed for 15 min following various treatments. Groups 1 and 2 were given a conventional dose of naloxone (10 mg/kg), while groups 3 and 4 were pretreated 30 min with either saline (3) or compound A3 (4, 100 mg/kg, s.c.) ahead of administration of low-dose naloxone (0.1 mg/kg). Immediately after the various naloxone treatments, withdrawal behaviors were quantified from mice in 16×45 cm plexiglass cylinders for 15 minutes following established protocols^{42, 43}.

### Pharmacokinetic Studies

Compound A3 was administered to mice intravenously at 10 mg/kg dose in four C57BL/6J mice for each time point. After 30, 60, or 120 minutes, mice were anesthetized with isoflurane followed by removal of blood and the animals were sacrificed for removal of the brain. Brains were rinsed with PBS, dried, flash-frozen and weighed for subsequent studies on brain penetration of compound A3. Tissue samples were placed into Navy bead lysis kit tubes and compared against naïve tissue, which was used for standard, quality control and blank samples. An appropriate volume of cold acetonitrile:water (3:1) was added to each sample tube to normalize the concentrations to 250 mg/mL. Tubes were then placed into a bead heater for 3 minutes, followed by centrifugation at 3200 rpm for 5 minutes at 4° C. The resulting supernatants were transferred to Eppendorf tubes and stored at −80° C. until analysis.

For analysis, samples were thawed on ice and extensively mixed, followed by further centrifugation at 3200 rpm for 5 minutes at 4° C. 30 μL of supernatant was collected and transferred to a 96 well plate along with 30 μL standards, quality controls, blanks, and double blanks. Cold acetonitrile (150 μL) spiked with internal standard was then added to blanks, standards, quality controls and unknown samples, while cold acetonitrile alone was added to double blanks. Samples were mixed for 10 minutes followed by further centrifugation at 3200 rpm for 10 minutes at 4° C. Supernatants after centrifugation were evaporated to dryness in a 96 well plate, followed by reconstitution with 100 μL 0.1% v/v formic acid in water:acetonitrile (90:10). The plate was sealed, vortexed, and briefly centrifuged to settle the samples, and was submitted for LC/MS analysis as previously described^{11, 34} using a mass spectrometer (SCIEX Triple Quad 5500+ system—QTRAP ready) linked to a liquid chromatography system (ExionLC AD-HPLC).

### Antinociceptive Testing

The 55° C. warm-water tail-withdrawal assay was performed as previously described^{42, 47}. Latency of the mouse to remove its tail from warm water was recorded as the endpoint. After determining baseline tail-withdrawal latencies, mice were administered opioid agonists with or without subcutaneous pretreatment with vehicle (10% DMSO/10% Solutol/80% saline, 0.9%), a graded dose of compound A3 (1-100 mg/kg) and/or naloxone. Vehicle or compound A3 was administered 30 minutes before administering saline or naloxone (0.1, 1 or 10 mg/kg) 15 min in advance of morphine (10 mg/kg, s.c), at which point the tail-withdrawal assay was performed every 10 minutes until a return to baseline responses were achieved (out to 150 minutes). To avoid excess tissue damage, mice that failed to withdraw their tails after 15 seconds had their tails removed from the bath and were assigned a maximum antinociceptive response (100%, see below equation). The assay was additionally performed with fentanyl as the orthosteric opioid agonist, but fentanyl was administered at 0.4 mg/kg. At each time point, antinociception was then calculated as follows:

$$\% antinociception = 100 * ( ( test latency - baseline latency ) / ( 15 - b aseline latrncy ) )$$

### Locomotor and Respiration Effects

Locomotor activity (as ambulations) and respiratory depression (as breaths per minute) were measured using automated, closed-air Comprehensive Lab Animal Monitoring system (CLAMS) in a similar manner to that previously described^{42, 43, 48}. Briefly, mice were habituated in chambers for 60 minutes, during which baselines for each animal were measured. Vehicle or increasing doses of compound A3 (1-100 mg/kg) was then administered, followed 30 min later by administration of either vehicle, morphine (10 mg/kg), or morphine with low-dose naloxone (0.1 mg/kg), and confined 5 min later in individual CLAMS testing chambers for measures lasting 200 minutes. The respiration rate (breaths per minute) was measured via pressure transducers built into the sealed CLAMS cages, whereas infrared beams located in the cage floors measured ambulations via sequential beam breaks. Data are expressed as a percentage of vehicle responses±SEM for ambulations or breaths per minute, averaged over 20-min periods for 140 min post-injection of the test compound.

### Results

### DNA-Encoded Library Screen for MOR Allosteric Modulators

In order to identify PAMs that could enhance analgesic effects induced by met-enkephalin, one of the most abundant opioid peptides in the brain^{8, 24}, excess met-enkephalin peptide was bound to the MOR receptor and formed active, nucleotide-free complex with a predominant G protein signaling partner of the MOR, Gᵢ₁. Any DEL components enriched in this condition are then likely to bind 1) to the active ensemble of the receptor and 2) in a site distinct and non-competitive with enkephalin. Conversely, to discover NAMs that may inhibit activation of the receptor, DEL components were selected for that bind specifically to MOR saturated with naloxone. These two conditions then serve as counter-selections for each other; selective enrichment in one and not the other indicates that molecule is indeed sensitive to the activation state of the receptor.

In order to identify potential PAMs of CB1, compounds were selected for that interacted with the active cannabinoid receptor 1 (CB1) bound to the potent full agonist MDMB-Fubinaca²⁵ (FUB) and the same Gᵢ₁ used for MOR. This condition then serves as a positive selection for CB1PAMs, as well as serving as a counter-selection with the MOR/Gᵢ₁ sample to remove Gᵢ₁-specific binders. Finally, no target control eliminates molecules that bind non-specifically to the beads used to immobilize receptors.

After several rounds of binding the DEL to all 4 conditions, washing away unbound components between, the enriched components were subjected to next-generation sequencing and relative enrichment scores were calculated based on the number of sequence reads in each condition and normalizing to the unselected library. There were several “families” of enriched compounds in all conditions, with enrichment values (and raw sequence counts) ranging from moderate 634 (24) to very highly enriched >100,000 (595). From families of enriched compounds, we then selected the smallest, most polar representative compounds, including examples from three MOR PAM families, one MOR NAM family, and one CB1PAM family (FIG. 1). Compounds were then screened biochemically using an in vitro GTP-turnover assay^{26, 27} where we assessed their ability to enhance (PAM) or dampen (NAM) agonist-dependent GTP depletion. Two of the three compounds selected as prospective MOR PAMs because of their specific enrichment to active MOR do indeed enhance met-enkephalin induced activation of G protein, A1 and A2. A5 does not appear to modulate signaling of the receptor.

The mechanism of action of A1 was characterized because it is more efficacious at improving GTP turnover compared to A2. Moreover, it shares a similar potency while being smaller. Further, the compound selected as a potential MOR NAM due to its extremely high and specific enrichment against inactive MOR (A3) significantly inhibits met-enkephalin induced activation of Gᵢ (FIG. 1D). Finally, A4 was selected as a potential CB1PAM. It does indeed enhance FUB-induced Gᵢ turnover. None of the molecules exert their effects in the absence of receptor. A4 is a comparatively small compound (446 Da) composed of 2 DEL fragments (rather than 3 or 4 as in the MOR compounds) and is much more polar than other CB1PAM compounds with a predicted log P value of 2.62.

### MOR PAM and NAM Activity of Compound A1 and Compound A3

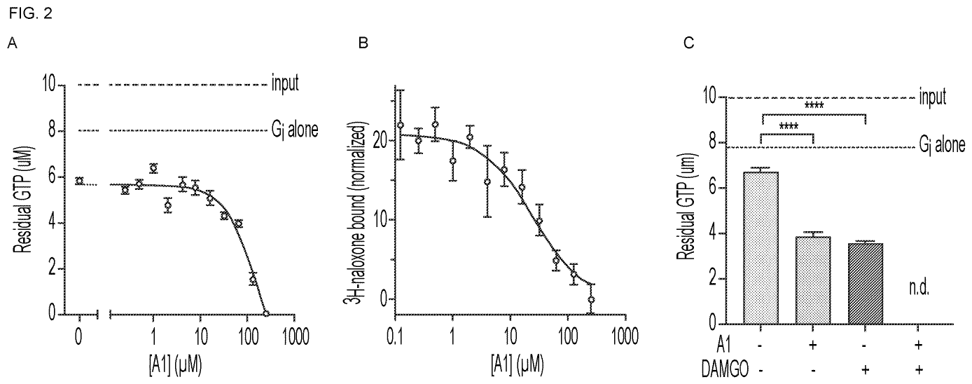

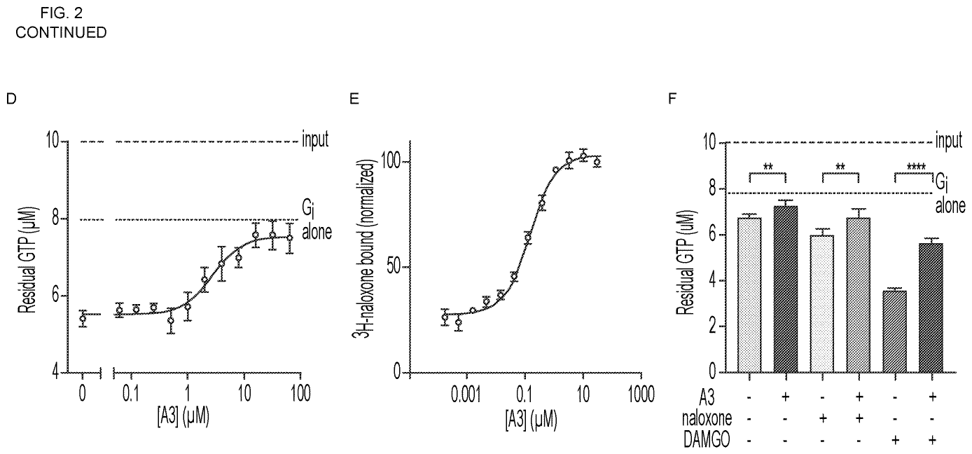

A1 enhances met-enkephalin-induced turnover with a low double-digit micromolar potency as assessed by the GTP turnover assay (FIG. 2A). Though it has a low potency, it is highly effective at enhancing activity, fully depleting nucleotide under these conditions and converting met-enkephalin into a super agonist. Consistent with its binding to (and presumably stabilizing) the active ensemble of MOR conformations, increasing amounts of A1 decrease binding of ³H-naloxone to MOR-expressing membranes, with a comparable potency to that observed in the GTP turnover assay (FIG. 2B). This decrease in ³H-naloxone binding is due to a ~6-fold decrease in naloxone affinity for the receptor in the presence of A1. In addition to its effect on endogenous opioid peptides, A1 enhances the efficacy of all orthosteric agonists including the very weak partial/neutral antagonist naloxone and partial agonists (MP), full agonist peptides (met-enkephalin, DAMGO), and potent small molecule full agonists (BU72). Further, A1 on its own, in the absence of orthosteric agonist, can induce GTP turnover to similar levels observed for the full agonist DAMGO, classifying it as an agonistic-PAM (ago-PAM) (FIG. 2C).

A3 was selected as a potential NAM for the MOR based upon its selective (and large) enrichment to only the inactive, naloxone-bound receptor with no enrichment observed to the active MOR-Gᵢ-met-enkephalin complex. Using the GTP-depletion assay, it was shown that A3 is >10-fold more potent at inhibiting MOR/met-enkephalin induced activation of G than A1 is at enhancing activation (FIG. 2D). The effect saturates at ~10 μM A3, with the result of inhibiting GTP depletion to nearly the same levels as those observed for G in the absence of any receptor catalyst. To more quantitatively assess the potency of A3, its effect on ³H-naloxone binding to MOR-expressing membranes was observed. Consistent with its selective enrichment to inactive, naloxone-bound receptor and its inhibition of MOR induced inhibition of Gᵢ signaling, titrating A3 results in increased binding of ³H-naloxone with an EC₅₀ of 130 nM (FIG. 2E). This increase in binding is the result of a ~2.6 fold increase in ³H-naloxone affinity in the presence of excess A3. This cooperativity with naloxone binding is also a direct demonstration of the allosteric mechanism of action of the compound, at least with respect to naloxone and potentially other morphinan ligands. A3 inhibits turnover of the receptor in the absence of orthosteric ligand (FIG. 2F), designating the molecule as the first opioid ligand able to efficiently inhibit basal signaling of the receptor. It is also able to inhibit additional turnover caused by the extremely weak partial agonist/neutral antagonist naloxone as well that from full agonist DAMGO (FIG. 2F). In addition to inhibiting turnover of the MOR, A3 enhances the observed on-rate of ³H-naloxone by >10-fold when the orthosteric site is saturated with high-affinity MOR agonist mitragynine pseudoindoxyl. Because orthosteric agonist off-rate is the limiting step in this experiment, this demonstrates the ability of A3 to actively enhance the off-rate of orthosteric agonist.

### CB1PAM Activity of A4

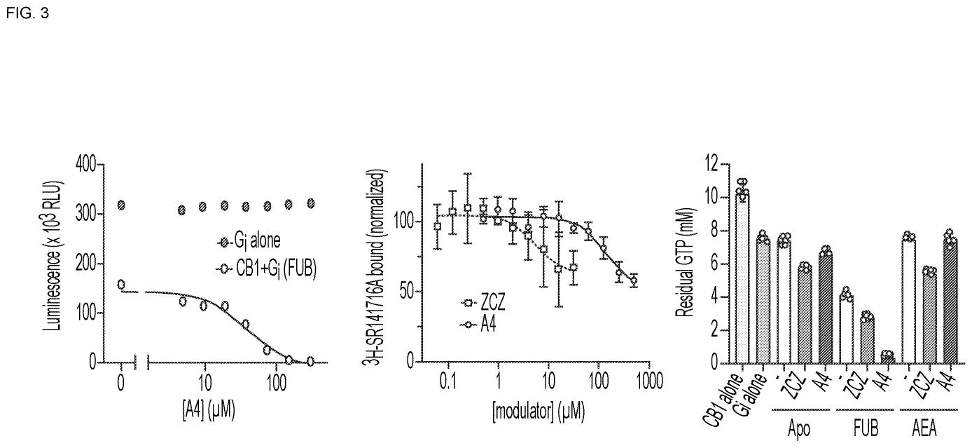

The biochemical GTP turnover assay demonstrates that A4 acts as a PAM in the presence of the synthetic cannabinoid FUB. In the presence of FUB-bound CB1, increasing the concentration of A4 results in the decrease of luminescence, indicating increasing GTP turnover of Gᵢ₁ (FIG. 3A). This shows that A4 is indeed a PAM as its presence further increases the turnover of Gᵢ₁ compared to FUB alone. Radioligand binding assay using the antagonist, ³H-SR141716A confirmed that A4 binds CB1 and decreases antagonist affinity though with lower potency than the known PAM, ZCZ (FIG. 3B). To investigate if A4 is able to increase turnover of all orthosteric ligands equally, a GTP turnover assay was performed in the presence of the synthetic cannabinoid, FUB and the eCB, AEA. The data showed that A4 is specifically able to increase turnover for FUB-bound CB1, and not AEA-bound CB1 (FIG. 3C). Comparatively, ZCZ causes increased Gᵢ₁ turnover for both FUB and AEA, equally. Hence, A4 is a PAM specifically for FUB, which was used for DEL selections.

### Synthesis of Compound B1

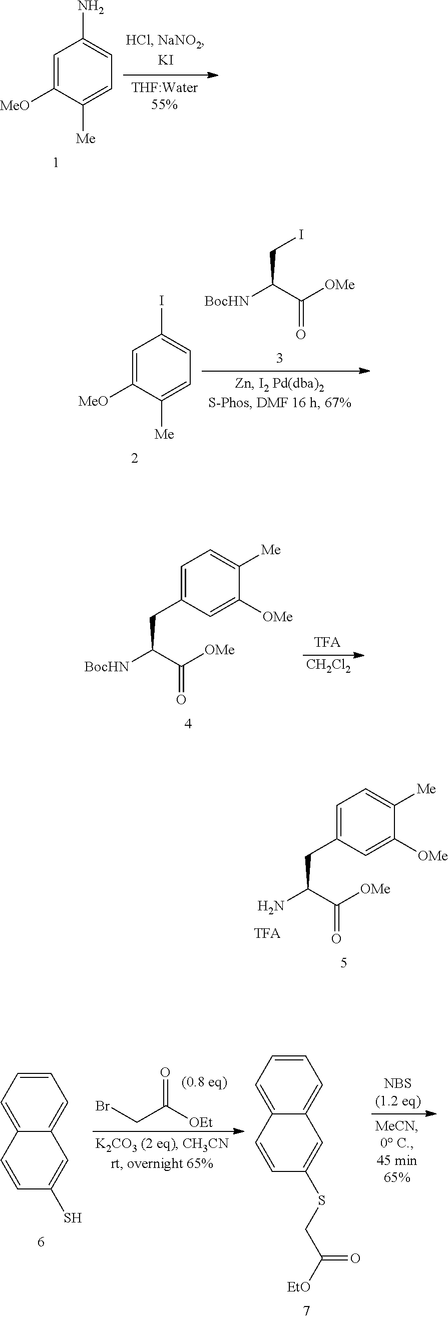

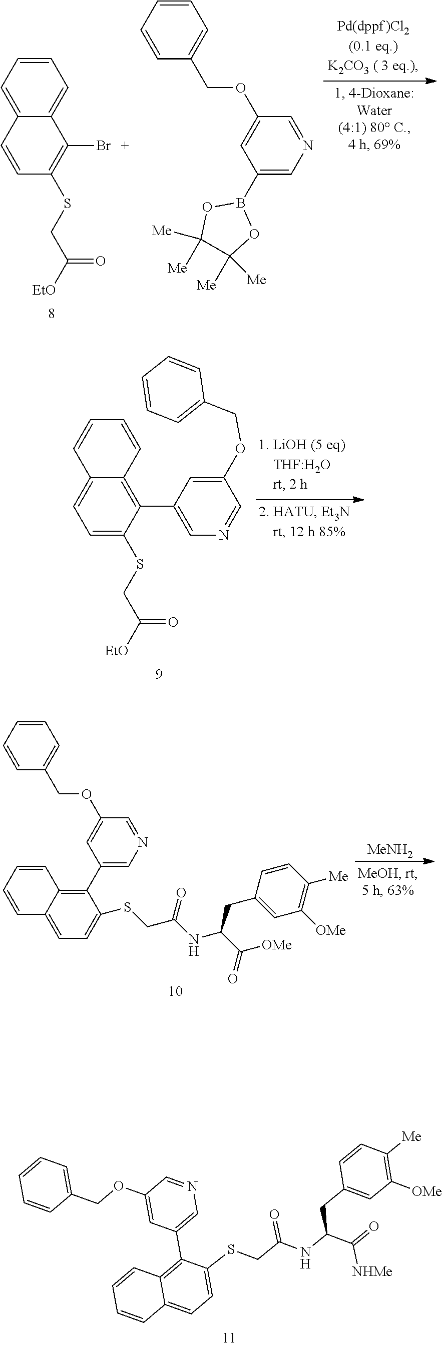

Compound B1 (11) was synthesized according the scheme above.

Step 1. 4-iodo-2-methoxy-1-methylbenzene (2): To a stirred solution of Compound 1 (5 g, 36.49 mmol) in (50 mL) THE was added 15 mL HCl in (25 mL) H₂O at 0° C. NaNO₂ (3.02 g, 43.79 mmol) solution in (10 mL) was added dropwise and reaction mixture was stirred at the same temperature for 20 minutes. A solution of KI in (50 mL) of water was added slowly to the reaction mixture at 0° C. and stirred at room temperature for another 18 hours. The reaction mixture was diluted with water and EtOAC (100 mL), the organic layer was separated and aqueous layer was extracted with EtOAc (100 mL×2). The combined organic layer was washed with saturated 10% NaOH (50 mL), and saturated Na₂S₂O₃ and water. The organic layer was dried over anhydrous Na₂SO₄ and evaporated off and the crude reaction mixture was purified by flash chromatography using 1-5% EtOAc:Hexane yielding the iodo compound 2 (4.5 g). ¹H NMR (500 MHz, CDCl₃) δ 7.16 (d, J=7.7 Hz, 1H), 7.07 (s, 1H), 6.82 (d, J=7.7 Hz, 1H), 3.78 (s, 3H), 2.13 (s, 3H). ¹³C NMR (126 MHz, CDCl₃) δ 158.3, 132.0, 129.4, 126.5, 119.2, 90.4, 55.5, 15.9.

Step 2. Methyl (S)-2-((tert-butoxycarbonyl)amino)-3-(3-methoxy-4-methylphenyl)propanoate (4): Zinc (0.193 g, 0.80 mmol) was dissolved in DMF (2 mL) to it I₂ (0.023 g, 0.090 mmol) was added under N₂. Compound 3 (0.2 g, 0.60 mmol) and iodine (0.023 g, 0.090 mmol) was added. The reaction was stirred at room temperature for 5 minutes. To this Pd(dba)₃ (13.8 mg, 0.015 mmol), S-Phos (12 mg, 0.03 mmol) and 4-iodo-2-methoxy-1-methylbenzene (0.193 g, 0.80 mmol) were added sequentially, and reaction mixture was stirred at room temperature overnight. After, the reaction mixture was diluted with DCM (10 mL) and cold water (10 mL), the organic layer was separated, and the aqueous layer was extracted with DCM (10 mL×2). The organic layers were combined washed with water, brine and dried over anhydrous Na₂SO₄ The volatile solvents were evaporated off to furnish crude residue which was purified by flash column chromatography (using 5-10%, EtOAc:hexane) to produce compound 4 (0.130 g).¹H NMR (500 MHz, CDCl₃) δ 7.04 (d, J=7.3 Hz, 1H), 6.69-6.55 (m, 2H), 4.97 (s, 1H), 4.63-4.51 (m, 1H), 3.81 (s, 3H), 3.73 (s, 3H), 3.17-2.94 (m, 2H), 2.18 (s, 3H), 1.43 (s, 9H). ¹³C NMR (126 MHz, CDCl₃) δ 172.4, 156.2, 134.6, 130.6, 121.1, 110.9, 81.9, 55.2, 54.5, 52.4, 38.2, 28.3, 15.9.

Step 3. methyl (S)-2-amino-3-(3-methoxy-4-methylphenyl)propanoate (5): Compound 4 was dissolved in CH₂Cl₂, followed by TFA. The solution was worked up according to any method commonly used in the art to provide compound 5 which used in next step without purification.

Step 4. Ethyl 2-(naphthalen-2-ylthio)acetate (7): Naphthalene-2-thiol (6) (1 g, 6.20 mmol) was dissolved in CH₃CN (20 mL) at room temperature to it K₂CO₃ (1.72 g, 12.4 mmol) and ethyl bromoacetate (55 mL, 5 mmol) was added and reaction was stirred at room temperature overnight. To this 0.5 N NaOH (20 mL) was added and diluted with EtOAc (20 mL). The organic layer was separated, and aqueous layer was extracted with EtOAc (20 mL×2). The organic layer was washed with brine and dried over anhydrous Na₂SO₄. The volatile solvents were evaporated off to furnish crude residue which was purified by flash column chromatography (using 1-5%, EtOAc:hexane) to provide compound 7 (0.8 g). ¹H NMR (500 MHz, CDCl₃) δ 7.98-7.64 (m, 4H), 7.49 (ddd, J=13.2, 7.0, 4.1 Hz, 3H), 4.18 (q, J=7.2 Hz, 2H), 3.75 (s, 2H), 1.22 (t, J=7.2 Hz, 3H).¹³C NMR (126 MHz, CDCl₃) δ 169.6, 133.7, 132.4, 132.1, 128.6, 128.2, 127.7, 127.6, 127.3, 126.6, 126.1, 61.6, 36.7, 14.1.

Step 5. Ethyl 2-((1-bromonaphthalen-2-yl)thio)acetate (8): Compound 7 (0.1, 0.4 mmol) was dissolved in CH₃CN (5 mL) and cooled to 0° C., NBS (0.086 g, 0.48 mmol) was added, and the reaction stirred at same temperature for 45 minutes. After completion of reaction, it was diluted with EtOAc (20 mL), and sat. NaHCO₃, and water. The organic layer was separated, and aqueous layer was extracted with EtOAc (10 mL×2). The combined organic layer was washed with brine and dried over anhydrous Na₂SO₄. The volatile solvents were evaporated off to furnish crude residue which was purified by flash column chromatography (using 1-5%, EtOAc:hexane) to provide compound 8 (0.088 g). ¹H NMR (500 MHz, CDCl₃) δ 8.24 (d, J=8.5 Hz, 1H), 7.81-7.72 (m, 2H), 7.58 (t, J=7.2 Hz, 1H), 7.53-7.44 (m, 2H), 4.19 (q, J=7.1 Hz, 2H), 3.79 (s, 2H), 1.23 (t, J=7.1 Hz, 3H). ¹³C NMR (126 MHz, CDCl₃) δ 169.1, 134.6, 132.62, 132.6 128.1, 127.9, 126.9, 126.3, 125.6, 123.3, 61.7, 35.8, 14.0.

Step 6. Ethyl 2-((1-(5-(benzyloxy)pyridin-3-yl)naphthalen-2-yl)thio)acetate (9): Compound 8 (0.02 g, 0.06 mol) was dissolved in 1,4-dioxane:water (2.5 mL, 4:1) and argon was bubbled in for 10 minutes. K₂CO₃ (0.025 g, 0.18 mmol) and ([1,1′-Bis(diphenylphosphino)ferrocene]dichloropalladium(II)) (0.005 g, 0.001 mmol) was added and reaction mixture was stirred under argon at 80° C. for 4 hours. After, the reaction mixture was dried over anhydrous Na₂SO₄ and filtered through celite pad, the celite pad washed with EtOAc (20 mL). The volatile solvents were evaporated off and the residue obtained purified by flash column chromatography to provide compound 9 (0.018 g, 69% yield). ¹H NMR (500 MHz, CDCl₃) δ 8.52 (d, J=2.3 Hz, 1H), 8.21 (s, 1H), 7.87 (dd, J=12.7, 8.8 Hz, 2H), 7.65 (d, J=8.7 Hz, 1H), 7.59-7.17 (m, 9H), 5.15 (s, 2H), 4.14 (q, J=7.2 Hz, 2H), 3.55 (s, 2H) 1.20 (t, J=7.1 Hz, 3H). ¹³C NMR (126 MHz, CDCl₃) δ 169.2, 154.5, 143.5, 137.7, 136.0, 135.7, 134.6, 132.8, 132.2, 132.1, 129.1, 128.6, 128.2, 128.0 (2C), 127.6, 127.0 (2C), 126.5, 126.0, 125.6, 123.6, 70.3, 61.5, 36.2, 14.0.

Step 7. Methyl(S)-2-(2-((1-(5-(benzyloxy)pyridin-3-yl)naphthalen-2-yl)thio)acetamido)-3-(3-methoxy-4-methylphenyl)propanoate (10): Compound 9 (0.05 g, 0.11 mol) was dissolved in THF:H₂O (3 mL, 2:1), to this LiOH (1N, 0.6 mL, 0.58 mmol) was added and reaction stirred overnight. After completion of reaction, it was acidified using 1N HCl and extracted with EtOAc (20 mL×2), the organic layer was washed with brine and dried over anhydrous Na₂SO₄. The volatile solvents were evaporated off to give the crude acid. Compound 4 (0.049 g, 0.15 mmol) was dissolved in CH₂Cl₂ (2 mL) to it TFA (0.5 mL) was added, and reaction stirred for 3 hours. After completion of reaction, the solvent was evaporated off and residue 5 dissolved in CH₂Cl₂:DMF (3 mL, 2:1) and to it the acid obtained above was added using DMF (1 mL) to this Et₃N (0.05 mL, 0.35 mmol), HATU (0.067 g, 0.17 mmol) was added sequentially, and reaction mixture was stirred at room temperature overnight. The reaction mixture was diluted with cold water (10 mL) and extracted with EtOAc (20 mL×2). The combined organic layer was washed with brine and dried over anhydrous Na₂SO₄. The volatile solvents were evaporated off to furnish crude residue which was purified by flash column chromatography using (1-50%, EtOAc:hexane) to provide compound 10 (0.06 g). ¹H NMR (500 MHz, CDCl₃) δ 8.58-8.44 (m, 1H), 8.12 (d, J=49.7 Hz, 1H), 7.91-7.78 (m, 2H), 7.54-7.25 (m, 12H), 7.04 (s, 1H), 6.39 (d, J=6.9 Hz, 1H), 5.15 (s, 1H), 5.06-4.94 (m, 1H), 4.85-4.67 (m, 1H), 3.72-3.60 (m, 5H), 3.57 (s, 3H), 3.09-2.96 (m, 2H), 1.99 (m, 3H). ¹³C NMR (100 MHz, CDCl₃) δ 171.5, 167.6, 157.7, 143.1, 135.8, 134.4, 133.9, 131.7, 130.5, 129.5, 128.7, 128.7, 128.4, 128.3, 128.2, 127.8, 127.7, 127.3, 126.0, 125.3, 125.2, 120.9, 120.6, 110.3, 70.4, 55.1, 53.4, 52.4, 37.5, 36.6, 15.7.

Step 8. (S)-2-(2-((1-(5-(benzyloxy)pyridin-3-yl)naphthalen-2-yl)thio)acetamido)-3-(3-methoxy-4-methylphenyl)-N-methylpropanamide (11): Compound 10 was dissolved in MeOH and to it MeNH₂ (40% in H₂O, 0.25 mL) was added and reaction stirred at room temperature overnight. After completion of reaction, volatile solvents were evaporated off and residue purified by flash chromatography using (80% EtOAc:hexane) to furnish the product compound 11 (7 mg). ¹H NMR (500 MHz, CDCl₃) δ 8.54 (s, 1H), 8.17 (d, J=9.0 Hz, 1H), 7.92-7.75 (m, 2H), 7.53-7.26 (m, 10H), 7.12 (s, 1H), 6.79 (dd, J=30.1, 7.3 Hz, 1H), 6.64-6.41 (m, 2H), 5.48 (d, J=24.3 Hz, 1H), 5.18 (ddt, J=24.7, 18.0, 9.3 Hz, 2H), 4.58-4.40 (m, 1H), 3.70 (s, 3H), 3.62-3.48 (m, 2H), 3.06-2.84 (m, 2H), 2.54 (s, 3H), 2.06 (s, 3H). ¹³C NMR (126 MHz, CDCl₃) δ 170.70 (d, J=4.0 Hz), 167.8, 157.9, 135.9, 135.0, 134.5, 131.9, 130.6, 129.6, 128.7, 128.3, 128.1, 127.71, 127.7, 127.4, 126.1, 125.4, 124.1, 124.0, 120.7, 110.6, 70.5, 55.2, 38.0, 37.2, 37.0, 36.6, 15.8. HRMS calcd for C₃₆H₃₅N₃O₄S Na+; 628.2240: HRMS found 628.2245.

### Synthesis of Compound B2

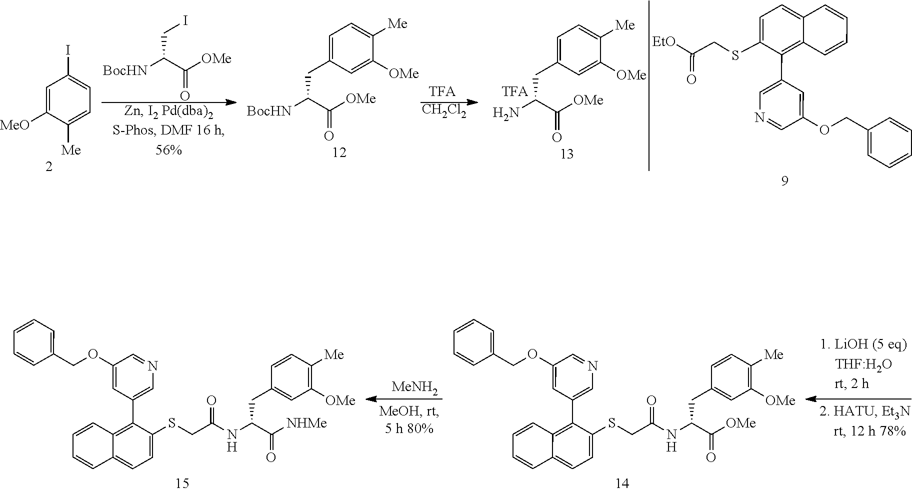

Compound B2 (15) was also synthesized (Scheme 2) following a similar protocol to the (S) isomer, compound B1.

Step 1. Methyl (R)-2-((tert-butoxycarbonyl)amino)-3-(3-methoxy-4-methylphenyl)propanoate (12): The synthetic procedure for compound 4 was followed with Methyl (S)-2-((tert-butoxycarbonyl)amino)-3-iodopropanoate (0.2 g, 0.60 mmol) to provide compound 12 (0.110 g, 56% yield). ¹H NMR (500 MHz, CDCl₃) δ 7.04 (d, J=7.3 Hz, 1H), 6.66-6.55 (m, 2H), 4.99 (s, 1H), 4.57 (s, 1H), 3.81 (s, 3H), 3.72 (s, 3H), 3.12-2.94 (m, 2H), 2.18 (s, 3H), 1.43 (s, 9H).

Step 2. methyl (R)-2-amino-3-(3-methoxy-4-methylphenyl)propanoate (13): Compound 12 was dissolved in CH₂Cl₂, followed by TFA. The solution was worked up according to any method commonly used in the art to provide compound 13 which used in next step without purification.

Step 3. Methyl(R)-2-(2-((1-(5-(benzyloxy)pyridin-3-yl)naphthalen-2-yl)thio)acetamido)-3-(3-methoxy-4-methylphenyl)propanoate (14): The synthetic procedure for compound 10 was followed, using compound 9 (0.05 g, 0.11 mmol) to provide compound 14 (0.055 g, 78% yield). ¹H NMR (500 MHz, CDCl₃) δ 8.43 (dd, J=5.6, 2.8 Hz, 1H), 7.84-7.68 (m, 2H), 7.53-7.15 (m, 10H), 7.09-6.88 (m, 2H), 6.58-6.34 (m, 1H), 6.34-6.22 (m, 2H), 5.10-01 (m, 1H), 4.93 (q, J=11.6 Hz, 1H), 4.75-4.61 (m, 1H), 3.57 (s, 3H), 3.54-3.51 (m, 2H), 3.49 (s, 3H), 307-2.83 (m, 2H), 1.90 (s, 3H). ¹³C NMR (126 MHz, CDCl₃) δ 171.5 167.6, 167.5, 157.6, 143.3, 138.1, 138.0, 136.0, 134.3, 134.0, 133.0, 132.8, 131.9, 131.8, 130.5, 129.5, 128.7, 128.0, 127.8, 127.7, 127.3, 126.0, 125.5, 123.7, 123.5, 123.0, 120.9, 120.7, 110.4, 70.4, 55.1, 53.5, 37.5, 36.9, 36.7, 15.7.

Step 4. (R)-2-(2-((1-(5-(benzyloxy)pyridin-3-yl)naphthalen-2-yl)thio)acetamido)-3-(3-methoxy-4-methylphenyl)-N-methylpropanamide (15): The synthetic procedure for 11 was performed with compound 14 to provide compound 15. ¹H NMR (500 MHz, CDCl₃) δ 8.53 (s, 1H), 8.16 (s, 1H), 7.94-7.72 (m, 2H), 7.50-7.28 (m, 10H), 7.13 (s, 1H), 6.86-6.72 (m, 1H), 6.63-6.39 (m, 2H), 5.46 (d, J=21.9 Hz, 1H), 5.29-5.07 (m, 2H), 4.59-4.43 (m, 1H), 3.70 (s, 3H), 3.64 (s, 2H), 3.06-2.83 (m, 2H), 2.54 (s, 3H), 2.06 (s, 3H). ¹³C NMR (126 MHz, CDCl₃) δ 170.7, 167.8, 157.8, 154.8, 143.5, 143.3, 138.15, 138.1, 136.0, 134.9, 134.6, 134.3, 132.9, 131.9, 131.7, 130.6, 129.5, 128.7, 128.3, 128.1, 127.7, 127.3, 126.1, 125.4, 124.0, 123.9, 123.5, 120.7, 110.6, 70.5, 55.2, 38.0, 37.19, 37.0, 26.1, 15.8. HRMS calcd for C₃₆H₃₅N₃O₄S Na+; 628.2240: HRMS found 628.2243

### Synthesis of Compound A3

Compound A3 (20) was prepared as well, following the scheme above.

Step 1. Methyl 2-((tert-butoxycarbonyl)amino)-3-(3-methoxy-4-methylphenyl)propanoate (16): The synthetic procedure for compound 4 was performed, but using methyl 2-((tert-butoxycarbonyl)amino)-3-iodopropanoate (0.2 g, 0.60 mmol) provided compound 16 (0.12 g, 61% yield). ¹H NMR (500 MHz, CDCl₃) δ 7.13-6.95 (m, 1H), 6.67-6.48 (m, 2H), 5.14-4.92 (m, 1H), 4.57 (d, J=7.8 Hz, 1H), 3.80 (s, 3H), 3.72 (s, 3H), 3.05 (dd, J=12.9, 6.0 Hz, 2H), 2.18 (s, 3H), 1.42 (s, 9H).¹³C NMR (126 MHz, CDCl₃) δ 172.4, 157.7, 155.1, 134.6, 130.6, 125.3, 121.0, 110.9, 79.8, 55.2, 54.5, 52.1, 38.2, 28.3 (3C), 15.8.

Step 2. methyl-2-amino-3-(3-methoxy-4-methylphenyl)propanoate (17): Compound 16 was dissolved in CH₂Cl₂, followed by TFA. The solution was worked up according to any method commonly used in the art to provide compound 17 which used in next step without purification.

Step 3. Methyl 2-(2-((1-bromonaphthalen-2-yl)thio)acetamido)-3-(3-methoxy-4-methylphenyl) propanoate (18): The synthetic procedure for compound 10 was used with ethyl 2-((1-bromonaphthalen-2-yl)thio)acetate (8) (0.22 g, 0.68 mmol) provided compound 18 (0.26 g, 76% yield). ¹H NMR (500 MHz, CDCl₃) δ 8.23 (dd, J=8.6, 1.0 Hz, 1H), 7.82-7.74 (m, 1H), 7.71-7.65 (m, 1H), 7.60 (ddd, J=8.4, 6.8, 1.3 Hz, 1H), 7.50 (ddd, J=8.1, 6.9, 1.2 Hz, 1H), 7.18 (dd, J=15.3, 8.4 Hz, 2H), 6.77-6.69 (m, 1H), 6.45 (d, J=1.7 Hz, 1H), 6.35 (dd, J=7.5, 1.7 Hz, 1H), 4.81 (ddd, J=7.8, 6.9, 5.4 Hz, 1H), 3.74 (s, 2H), 3.66 (s, 3H), 3.64 (s, 3H), 3.00 (qd, J=14.0, 6.2 Hz, 2H), 2.00 (s, 3H).¹³C NMR (126 MHz, CDCl₃) δ 171.4, 167.3, 157.7, 134.0, 133.8, 132.5, 132.4, 130.5, 128.4, 128.2, 128.1, 126.6, 126.3, 125.3, 123.7, 121.9, 120.6, 110.3, 55.1, 53.4, 52.2, 37.5, 36.8, 15.7.

Step 4. 2-(2-((1-bromonaphthalen-2-yl)thio)acetamido)-3-(3-methoxy-4-methylphenyl)-N-methylpropanamide (19): Methyl 2-(2-((1-bromonaphthalen-2-yl)thio)acetamido)-3-(3-methoxy-4-methylphenyl)propanoate (0.250 g, 0.49 mmol) was dissolved in MeOH:CH₂Cl₂ (10:1, 11 mL) to it NH₂Me (40% in H₂O, 5 mL) was added and reaction was stirred at room temperature for overnight. After, the volatile solvents were evaporated off to complete dryness and residue used in next step without purification.

Step 5. 2-(2-((1-(5-(benzyloxy)pyridin-3-yl)naphthalen-2-yl)thio)acetamido)-3-(3-methoxy-4-methylphenyl)-N-methylpropanamide (20): The synthetic procedure for compound 11 was followed, using compound 19 to provide compound 20. ¹H NMR (399 MHz, CDCl₃) δ 8.51 (t, J=2.7 Hz, 1H), 8.15 (s, 1H), 7.94-7.72 (m, 2H), 7.52-7.17 (m, 11H), 6.81-6.70 (m, 1H), 6.54 (m, 1H), 6.46-6.40 (m, 1H), 5.89-5.66 (m, 1H), 5.25-5.06 (m, 2H), 4.54-4.47 (m, 1H), 3.66 (s, 3H), 3.58-3.47 (m, 2H), 3.01-2.86 (m, 2H), 2.54 (s, 3H), 2.04 (s, 3H). ¹³C NMR (100 MHz, CDCl₃) δ 170.8, 168.0, 157.8, 154.7, 143.4, 143.2, 138.1, 138.0, 136.0, 135.0 134.5, 134.2, 132.9, 131.8, 131.7, 130.5, 129.4, 128.7, 128.63, 128.1, 127.7, 127.65, 127.3, 126.0, 125.3, 124.01, 123.3, 120.7, 110.6, 70.4, 55.1, 54.9, 38.0, 37.1, 26.0, 15.7. HRMS calcd for C₃₆H₃₅N₃O₄S Na+; 628.2240: HRMS found 628.224

### Pharmacology of Compound A3

### GTP Turnover Assay

GTP-turnover assays were performed and showed that compound A3 inhibited MOR/met-enkephalin induced activation of Gₗ. The observed potency was in the single-digit μM range, though the concentration of receptor was on the same scale as compound A3, making quantitative evaluation of potencies difficult. The effect saturates at −10 μM compound A3, nearly eliminating receptor induced GTP turnover.

The potency of compound A3 was assessed quantitatively by ³H-naloxone binding experiments with MOR-expressing membranes. Titrating compound A3 results in increased binding of ³H-naloxone with an EC₅₀ of 133 nM (FIG. 2D). Consistent with its selective enrichment to inactive, naloxone-bound receptor and its inhibition of MOR induced inhibition of Gᵢ signaling, this increase in binding is the result of an increased affinity for ³H-naloxone, by a factor of ×2.6, in the presence of excess compound A3.

It was observed that compound A3 inhibited receptor-catalyzed GTP turnover in the absence of orthosteric ligands (FIG. 2F), indicative of the inverse agonist NAM (inverse ago-NAM) properties of compound A3 for the MOR. It was also shown to inhibit additional turnover caused by naloxone, a partial agonist/neutral antagonist, as well that from full agonist DAMGO (FIG. 2F). Overall, compound A3 resulted in inhibition of MOR-induced signaling when bound to a variety of orthosteric agonists, even when the concentrations of ligands present are in excess.

### TRUPATH Assay

The biochemical cooperativity observed between compound A3 and naloxone was studied in cells by performing a TRUPATH assay in antagonist mode for MOR-expressing cells. Naloxone was titrated in and the inhibition of C-protein heterotrimer dissociation was observed with bioluminescence resonance energy transfer (BRET). In the presence of compound A3, the observed potency of naloxone for inhibiting agonist-induced signaling was enhanced by a factor of ×7.6 (Table 1). Thus, the cooperativity between compound A3 and naloxone was observed in both biochemical and cellular environments. The observed cooperativity with naloxone binding also demonstrated the allosteric mechanism of action of compound A3.

TABLE 1
Summary of TRUPATH naloxone potency values

| | | pEC₅₀ ± SEM (BRET-Gi1 |
|---|---|---|
| Ligand | Compound A3 (μM) | protein activation), M |
| Naloxone | 0 | 7.82 ± 0.21 |
| | 90 | 8.69 ± 0.39 |

Potency (Log EC50, M) was derived from simultaneously fitting all biological replicates (n = 3 experiments each done in duplicate).

Next, a series of in-cell TRUPATH assays was conducted to test the effect of compound A3 on C-protein activation in cells. Several orthosteric agonists (morphine, fentanyl, and met-enkephalin) and all Ca-protein family members through which MOR signals (Cᵢ₁, Cᵢ₂, Cᵢ₃, C_{oA}, C_{oB}, and C_{z}) were studied. The observed potency in the TRUPATH assays for morphine, fentanyl, and met-enkephalin was decreased by a factor of ×10 or greater in the presence of compound A3 (Table 2). It was found that all C_{i/o} subtypes were inhibited nearly equally which indicated that there is no negative allosterism of compound A3 across C; family members.

TABLE 2
Summary of TRUPATH potency values for G_{i/o/z} activation

| | Compound | Orthosteric μOR agonists | | |
|---|---|---|---|---|
| pEC₅₀ ± SEM, | A3 | | | Met- |
| (M) | (μM) | Morphine | Fentanyl | enkephalin |
| Gαi1 | 0 | 8.29 ± 0.15 | 8.57 ± 0.11 | 8.64 ± 0.10 |
| | 90 | 7.09 ± 0.17 | 7.31 ± 0.10 | 7.44 ± 0.14 |
| Gαi2 | 0 | 8.01 ± 0.22 | 8.65 ± 0.17 | 8.61 ± 0.16 |
| | 90 | 6.95 ± 0.26 | 7.33 ± 0.23 | 7.26 ± 0.32 |
| Gαi3 | 0 | 8.06 ± 0.18 | 8.43 ± 0.15 | 8.43 ± 0.14 |
| | 90 | 6.72 ± 0.25 | 7.15 ± 0.12 | 7.28 ± 0.10 |
| GαoA | 0 | 8.40 ± 0.15 | 8.83 ± 0.11 | 8.82 ± 0.10 |
| | 90 | 7.28 ± 0.17 | 7.71 ± 0.19 | 7.60 ± 0.22 |
| GαoB | 0 | 8.47 ± 0.11 | 8.75 ± 0.10 | 8.89 ± 0.11 |
| | 90 | 7.34 ± 0.21 | 7.64 ± 0.15 | 7.75 ± 0.13 |
| GαZ | 0 | 8.56 ± 0.11 | 8.98 ± 0.10 | 9.12 ± 0.10 |
| | 90 | 7.48 ± 0.12 | 7.83 ± 0.14 | 7.97 ± 0.16 |

Potency (Log EC50, M) was derived from simultaneously fitting all biological replicates (n = 3 experiments each done in duplicate).

Next, the ability of compound A3 to inhibit signaling by opioid receptor subtypes (MOR, δOR and κOR) was studied using the TRUPATH assay. Increasing concentrations of compound A3 (up to 30 μM) resulted in a right-shift in DAMGO potency at the MOR by a factor of ×3.7 (Table 3), consistent with NAM activity. The NAM also caused a right-shift in DPDPE potency at SOR, though to a lesser extent (Table 3). No effect is observed at KOR activation by U50,488 (Table 3).

TABLE 3
Compound A3 subtype selectivity by TRUPATH assay

| | pEC₅₀ ± SEM (BRET-Gi1 protein activation), M | | | |
|---|---|---|---|---|
| | 0 μM | 3 μM | 10 μM | 30 μM |
| Receptor/ | Compound | Compound | Compound | Compound |
| ligand | A3 | A3 | A3 | A3 |
| MOR/DAMGO | 8.19 ± 0.06 | 8.11 ± 0.08 | 7.92 ± 0.09 | 7.62 ± 0.12 |
| δOR/DPDPE | 9.37 ± 0.05 | 9.24 ± 0.05 | 9.16 ± 0.06 | 8.91 ± 0.07 |
| κOR/U50488 | 8.77 ± 0.08 | 8.80 ± 0.08 | 8.68 ± 0.09 | 8.63 ± 0.11 |

Potency (Log EC50, M) was derived from simultaneously fitting all biological replicates (n = 3 experiments each done in duplicate).

### In-Vivo Study of Coadministration of Naloxone and Compound A3

Based on the substantial effects of the NAM on naloxone affinity and kinetics, the ability of compound A3 to potentiate naloxone in vivo was evaluated using mouse models. When administered intravenously at 10 mg/kg in mice, compound A3 was shown to enter the brain with moderate penetration (~25% of maximum plasma levels, which corresponded to an increase in the affinity of compound A3 by a factor of ×10) with a reasonable observed half-life (0.74 hours).

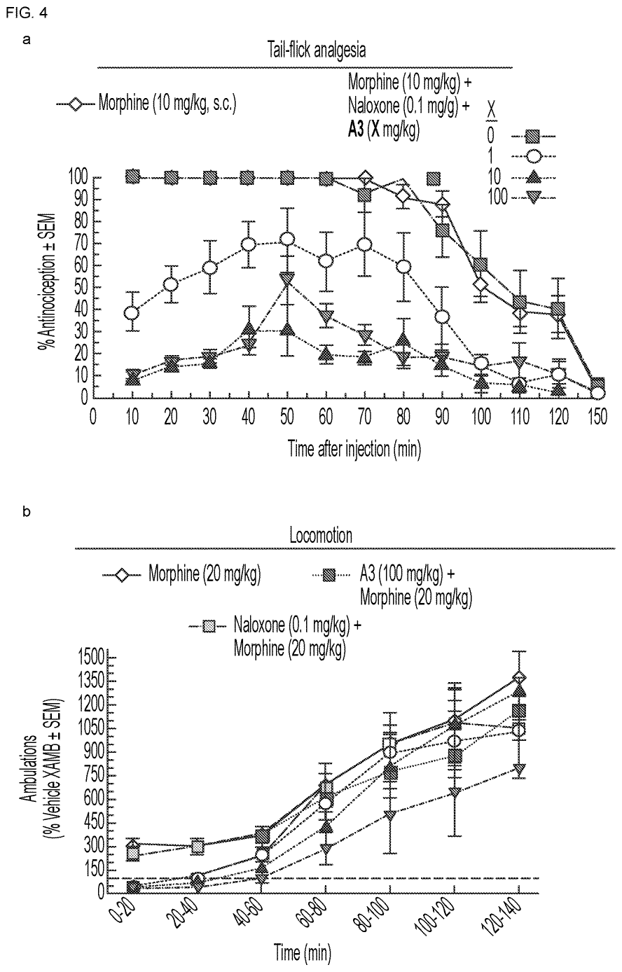

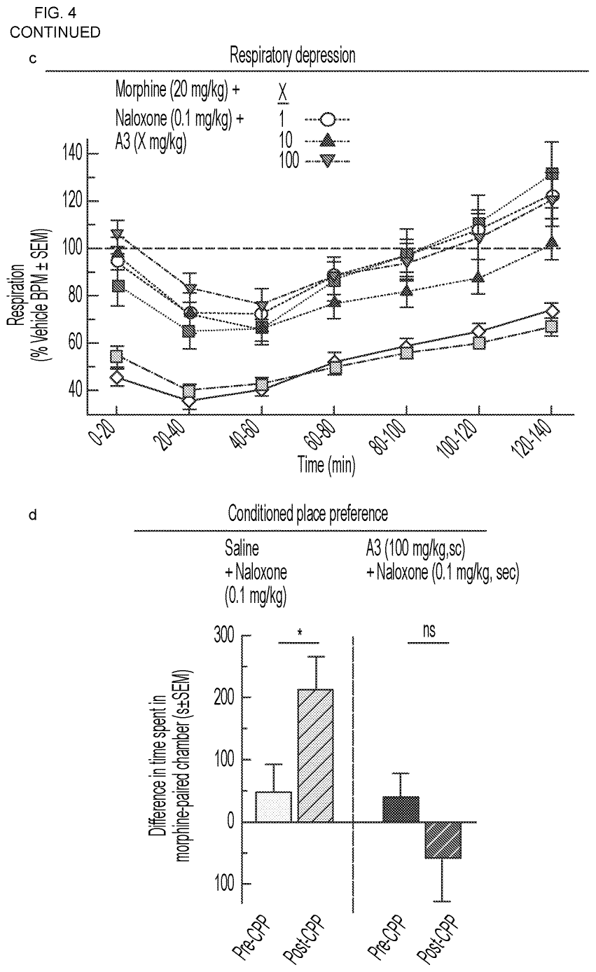

The ability of compound A3 to potentiate low-dose naloxone reversal of morphine induced antinociception was studied. Typically, naloxone is administered at doses of 1 or 10 mg/kg, s.c. to efficiently inhibit morphine-induced antinociception.^{40, 42, 43} However, the dose used (0.1 mg/kg s.c.) was confirmed to have no significant antagonism (FIG. 4A), while compound A3 alone has no significant impact on morphine-induced antinociception. In the presence of low-dose naloxone, compound A3 significantly inhibits morphine-induced antinociception in a dose-dependent manner (FIG. 4A). The median dose of compound A3 to potentiate low-dose naloxone antagonism of morphine antinociception was determined to be 1.15 (0.03-4.04) mg/kg, s.c.

Next, the ability of compound A3, in conjunction with the low (0.1 mg/kg) dose of naloxone, to inhibit morphine-mediated respiratory depression and hyperlocomotion was studied using the Comprehensive Lab Animal Monitoring System (CLAMS) assay.^{43, 44} Morphine (20 mg/kg, s.c.) significantly increased ambulations for over 140 min (FIG. 4B) and decreased breathing rate through 100 min (FIG. 4C). Pretreatment with the low dose of naloxone was ineffective in ameliorating morphine-induced hyperlocomotion (FIG. 4B), but was observed to reduce morphine-induced respiratory depression (FIG. 4C). While compound A3 itself does not change ambulation or breathing rate relative to vehicle administration, compound A3 significantly enhances low-dose naloxone inhibition of hyperlocomotion (FIG. 4B) and respiratory depression (FIG. 4C) induced by morphine, in a dose-dependent manner maximal at the 100 mg/kg dose. Although compound A3 substantially potentiates naloxone antagonism of morphine-induced antinociception and respiratory depression, this effect was not observed against fentanyl (0.4 mg/kg)-induced antinociception.

The combined effects of the NAM and a low-dose (0.1 mg/kg, s.c.) of naloxone were studied for inhibition of morphine-conditioned place preference (CPP).^{43, 44} While pretreatment of mice with vehicle (−30 min) and the low dose of naloxone (−15 min) alone did not prevent significant morphine CPP (FIG. 4D, left pair of bars), pretreatment with compound A3 (100 mg/kg, sc, −30 min) potentiated the MOR antagonism produced by the low dose of naloxone and morphine CPP was eliminated (FIG. 4D, right pair of bars).

Since compound A3 treatment potentiates low-doses of naloxone in reversing morphine effects in a wide array of assays (FIG. 4A, 4B, 4C, 4D), it was examined if compound A3 pretreatment potentiates naloxone-precipitated withdrawal symptoms^{41, 50}, This was performed by an assessment of the mice following administration of compound A3 (100 mg/kg, s.c.) and the low-dose of naloxone (0.1 mg/kg, s.c.) compared with the symptoms caused by treatment with conventional higher doses of naloxone (10 mg/kg, s.c.) alone. Assessment of withdrawal symptoms on the fifth day followed treatment with either the conventional high dose of naloxone, (10 mg/kg, group 2), the low-dose of naloxone used for experiments herein (0.1 mg/kg, group 3), or the low-dose of naloxone following treatment with compound A3 (100 mg/kg, group 4). The battery of withdrawal-associated behaviors after naloxone treatment (Table 4) demonstrated variable responses, but combined low-dose naloxone/NAM treatment did not potentiate the magnitude of withdrawal caused by a low-dose of naloxone alone to the same levels of withdrawal symptoms as the conventional naloxone treatment.

TABLE 4
Summary of behavioral endpoints of naloxone-precipitated withdrawal in saline-treated or
morphine-dependent mice following administration of either vehicle or 368 and naloxone (NAL)

| | | | | | | | Condition 4 | | |
|---|---|---|---|---|---|---|---|---|---|
| | | | | | Condition 3 | | morphine + | | |
| | Condition 1 | | Condition 2 | | morphine + low | | compound A3 + | | |
| | saline + NAL | | morphine + NAL | | NAL | | low NAL | | |
| Measure: | Avg | SEM | Avg | SEM | Avg | SEM | Avg | SEM | ANOVA result: |
| Forepaw Tremor | 26.7 | 12.4 | 26.7 | 12.4 | 7.3 | 2.381 | 1.6 | 1.6 | F_{(3,37)} = 2.05, |
| | | | | | | | | | p = 0.12 |
| Wet Dog shakes | 1 | 0.33 | 1.91 | 0.55 | 1 | 0.298 | 1.1 | 0.314 | F_{(3,37)} = 1.29, |
| | | | | | | | | | p = 0.29 |
| | | 0.1 | 0.55 | 0.31 | | 1.636 | | | F_{(3,37)} = 2.95, |
| Straightening | 0.1 | | | | 3.1 | | 0.2 | 0.133 | p = 0.05 |
| Stool | | | | | | | | | F_{(3,37)} = 23.8, |
| Consistency | 1.6 | 0.45 | 7.46* | 1.21 | 9.3* | 0.883 | 1.3+ | 0.367 | p < 0.0001 |
| Jumping | | | | | | | | | F_{(3,37)} = 17.0, |
| Frequency | 2.7 | 1.92 | 76.9* | 11.9 | 45.4*† | 6.578 | 32.9*† | 4.306 | p < 0.0001 |
| Rearing | | | | | | | | | F_{(3,37)} = 8.29, |
| Frequency | 23.5 | 7.54 | 2.64* | 1.25 | 22.2 | 3.699 | 1.6* | 0.686 | p = 0.0002 |
| Forepaw Licking | | | | | | | | | F_{(3,37)} = 3.88, |
| Frequency | 6† | 1.97 | 1.91 | 1.12 | 1.8 | 0.49 | 0.6 | 0.499 | p = 0.02 |
| Teeth Chattering | | | | | | | | | F_{(3,37)} = 12.8, |
| Frequency | 1 | 1 | 54.2* | 13.2 | 0.7+ | 0.3 | 10.9† | 2.858 | p < 0.0001 |

Average and Standard Error of the Mean for 5-day treatment with saline or morphine, followed by naloxone (condition 1 and 2 at 10 mg/kg, or a low dose of 0.1 mg/kg for condition 3 and 4) with or without compound A3 pretreatment (100 mg/kg, condition 3). One-way ANOVA analysis was performed using Tukey's post-hoc test with *p<0.05 versus Saline control, †p<0.05 versus Morphine+NaI (10 mg/kg) control. n=10-11/group.

For some behaviors (e.g. teeth chattering frequency), low-dose naloxone treatment alone had no effect relative to saline alone (P=0.76), and the addition of compound A3 increases withdrawal symptoms relative to both (P<0.0001, Tukey's post hoc test following one-way ANOVA), though to a lesser extent than the response to high-dose naloxone treatment (P<0.0001). For other measures, such as jumping frequency, low-dose naloxone treatment alone caused significant withdrawal symptoms, which is not altered in the presence of compound A3 (P=0.66). Of interest, while low- and high-dose naloxone treatment precipitates significant diarrhea in morphine-dependent mice, low-dose naloxone in combination with compound A3 treatment did not significantly change stool consistency relative to saline control (P=0.61).

### Stereoisomers of Compound A3

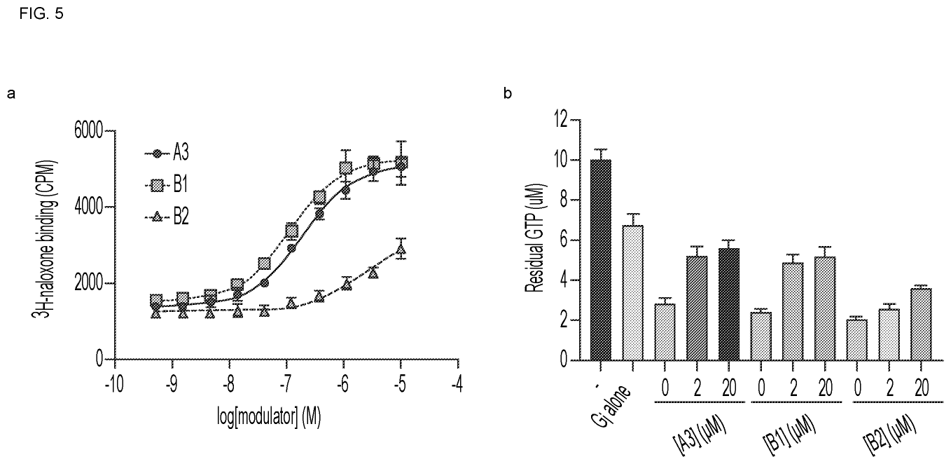

The effect of the chirality of compound A3 was studied with (S)-2-(2-((1-(5-(benzyloxy)pyridin-3-yl)naphthalen-2-yl)thio)acetamido)-3-(3-methoxy-4-methylphenyl)-N-methylpropanamide (Compound B1) and (R)-2-(2-((1-(5-(benzyloxy)pyridin-3-yl)naphthalen-2-yl)thio)acetamido)-3-(3-methoxy-4-methylphenyl)-N-methylpropanamide (Compound B2). Comparisons of ³H-naloxone results (FIG. 5A) and GTP turnover assays (FIG. 5B) showed that compound B1 was more potent than compound B2, by a factor of ×100 or more. The GTP turnover assay data also showed that compound B1 retains the ability to potently inhibit fentanyl (5 μM)-induced GTP turnover like the racemic compound A3, while compound B2 does not display full inhibition even at 20 μM.

### REFERENCES

1. Jamison, R. N. & Mao, J. Opioid Analgesics. Mayo Clin. Proc. 90, 957-968 (2015).
2. Centers for Disease Control and Prevention (CDC). Vital signs: overdoses of prescription opioid pain relievers—United States, 1999-2008. MMWR Morb. Mortal. Wkly. Rep. 60, 1487-1492 (2011).
3. Rudd, R. A. et al. Increases in heroin overdose deaths—28 states, 2010 to 2012. MMWR Morb. Mortal. Wkly. Rep. 63, 849-854 (2014).
4. O'Donnell, J. K., Halpin, J., Mattson, C. L., Goldberger, B. A. & Gladden, R. M. Deaths involving fentanyl, fentanyl analogs, and U-47700-10 states, July-December 2016. MMWR Morb. Mortal. Wkly. Rep. 66, 1197-1202 (2017).
5. Pain Management and the Opioid Epidemic: Balancing Societal and Individual Benefits and Risks of Prescription Opioid Use. National Academies Press (US) (2017). doi:10.17226/24781
6. Abdelal, R. et al. Real-world study of multiple naloxone administration for opioid overdose reversal among bystanders. Harm Reduct. J. 19, 1-10 (2022).
7. Thompson, G. L. et al. Biased agonism of endogenous opioid peptides at the μ-opioid receptor. Mol. Pharmacol. 88, 335-346 (2015).
8. Benarroch, E. E. Endogenous opioid systems: Current concepts and clinical correlations. Neurology 79, 807-814 (2012).
9. De Neve, J. et al. Comprehensive overview of biased pharmacology at the opioid receptors: biased ligands and bias factors. RSC Med. Chem. 12, 828-870 (2021).
10. Rasmussen, K., White, D. A. & Acri, J. B. NIDA's medication development priorities in response to the Opioid Crisis: ten most wanted. Neuropsychopharmacology 44, 657-659 (2019).
11. Lane, J. R., May, L. T., Parton, R. G., Sexton, P. M. & Christopoulos, A. A kinetic view of GPCR allostery and biased agonism. Nat. Chem. Biol. 13, 929-937 (2017).
12. Ibsen, M. S., Connor, M. & Glass, M. Cannabinoid CB1 and CB2 Receptor Signaling and Bias. Cannabis cannabinoid Res. 2, 48-60 (2017).
13. Wootten, D., Christopoulos, A., Marti-Solano, M., Babu, M. M. & Sexton, P. M. Mechanisms of signalling and biased agonism in G protein-coupled receptors. Nat. Rev. Mol. Cell Biol. 19, 638-653 (2018).
14. Slosky, L. M., Caron, M. G. & Barak, L. S. Biased Allosteric Modulators: New Frontiers in GPCR Drug Discovery. Trends Pharmacol. Sci. 42, 283-299 (2021).
15. Burford, N. T. et al. Discovery of positive allosteric modulators and silent allosteric modulators of the μ-opioid receptor. Proc. Natl. Acad. Sci. U.S.A 110, 10830-10835 (2013).
16. Bisignano, P. et al. Ligand-Based Discovery of a New Scaffold for Allosteric Modulation of the μ-Opioid Receptor. J. Chem. Inf. Model. 55, 1836-1843 (2015).
17. Pryce, K. D. et al. A Promising Chemical Series of Positive Allosteric Modulators of the μ-Opioid Receptor that Enhance the Antinociceptive Efficacy of Opioids but not their Adverse Effects. Neuropharmacology 195, 108673 (2021).
18. Vaysse, P. J.-J., Gardner, E. L. & Zukin, S. Modulation of Rat Brain Opioid Receptors by Cannabinoids. J. Pharmacol. Exp. Ther. 241, 534-539 (1987).
19. Rothman, R. B. et al. Salvinorin A: Allosteric interactions at the μ-opioid receptor. J. Pharmacol. Exp. Ther. 320, 801-810 (2007).
20. Tamba, B. I. et al. Challenges and opportunities in preclinical research of synthetic cannabinoids for pain therapy. Med. 56, 1-22 (2020).
21. Laprairie, R. B., Bagher, A. M., Kelly, M. E. M., Dupré, D. J. & Denovan-Wright, E. M. Type 1 cannabinoid receptor ligands display functional selectivity in a cell culture model of striatal medium spiny projection neurons. J. Biol. Chem. 289, 24845-24862 (2014).
22. Ignatowska-Jankowska, B. M. et al. A Cannabinoid CB 1 Receptor-Positive Allosteric Modulator Reduces Neuropathic Pain in the Mouse with No Psychoactive Effects. Neuropsychopharmacology 40, 2948-2959 (2015).
23. Franzini, R. M. & Randolph, C. Chemical space of DNA-encoded libraries: Miniperspective. J. Med. Chem. 59, 6629-6644 (2016).
24. Le Merrer, J., Becker, J. A. J., Befort, K. & Kieffer, B. L. Reward processing by the opioid system in the brain. Physiol. Rev. 89, 1379-1412 (2009).
25. Kumar, K. K. et al. Structure of a Signaling Cannabinoid Receptor 1-G Article Structure of a Signaling Cannabinoid Receptor 1-G Protein Complex. Cell 1-11 (2019). doi:10.1016/j.cell.2018.11.040
26. Gregorio, G. G. et al. Single-molecule analysis of ligand efficacy in ρ2AR-G-protein activation. Nature 547, 68-73 (2017).
27. Hilger, D. et al. Structural insights into differences in G protein activation by family A and family B GPCRs. Science 369, eaba3373 (2020).
28. Kaneko, S. et al. Activation mechanism of the μ-opioid receptor by an allosteric modulator. 1-10 (2022). doi:10.1073/pnas.2121918119/-/DCSupplemental.Published
29. Koehl, A. et al. Structure of the μ Opioid Receptor-G i Protein Complex. Nature 1-23 (2018). doi:10.1038/s41586-018-0219-7
30. Przewlocki, R. Opioid peptides. Neurosci. 21st Century From Basic to Clin. Second Ed. 1783-1810 (2016). doi:10.1007/978-1-4939-3474-4_54
31. Wang, H. et al. Structure-Based Evolution of G Protein-Biased μ-Opioid Receptor Agonists. Angew. Chemie—nt. Ed. (2022). doi:10.1002/anie.202200269
32. Robertson, M. J., van Zundert, G. C. P., Borrelli, K. & Skiniotis, G. GemSpot: A Pipeline for Robust Modeling of Ligands into Cryo-EM Maps. Structure 28, 707-716.e3 (2020).
33. Livingston, K. E. & Traynor, J. R. Allostery at opioid receptors: modulation with small molecule ligands. Br. J. Pharmacol. 175, 2846-2856 (2018).
34. Che, T. et al. Structure of the Nanobody-Stabilized Active State of the Kappa Opioid Receptor. Cell 172, 55-67.e15 (2018).
35. Che, T. et al. Nanobody-enabled monitoring of kappa opioid receptor states. Nat. Commun. 11, (2020).
36. Robertson, M. J. et al. Structure determinatino of inactive-state GPCRs with a universal nanobody. bioRxiv (2021).
37. Manglik, A. et al. Crystal structure of the μ-opioid receptor bound to a morphinan antagonist. Nature 485, 321-326 (2012).
38. Woodhams, S. G., Chapman, V., Finn, D. P., Hohmann, A. G., & Neugebauer, V. The cannabinoid system and pain. Neuropharmacology 124, 105-120 (2017).
39. Olsen, R. H. J. et al. TRUPATH, an open-source biosensor platform for interrogating the GPCR transducerome. Nat. Chem. Biol. 16, (2020).
40. Bloom, A. S., Dewey, W. L., Harris, L. S. & Brosius, K. K. The correlation between antinociceptive activity of narcotics and their antagonists as measured in the mouse tail-flick test and increased synthesis of brain catecholamines. J. Pharmacol. Exp. Ther. 198, 33-41 (1976).
41. Ryckaert, J.-P., Ciccotti, G. & Berendsen, H. J. C. Numerical integration of the Cartesian Equations of Motion of a System with Constraints: Molecular Dynamics of n-Alkanes. J. Comput. Phys. 23, 327-341 (1977).
42. Székely, J. I., Dunai-Kovács, Z., Miglécz, E., Ronai, A. Z. & Bajusz, S. In vivo antagonism by naloxone of morphine, beta-endorphin and a synthetic enkephalin analog. J. Pharmacol. Exp. Ther. 207, 878-883 (1978).
43. Chakraborty, S. et al. Oxidative Metabolism as a Modulator of Kratom's Biological Actions. J. Med. Chem. 64, 16553-16572 (2021).
44. Faouzi, A. et al. Structure-based design of bitopic ligands for the μ-opioid receptor. Nature 613, 767-774 (2023).
45. Kilkenny, C., Browne, W. J., Cuthill, I. C., Emerson, M. & Altman, D. G. Improving bioscience research reporting: The arrive guidelines for reporting animal research. Animals 4, 35-44 (2014).
46. Chakraborty, S. et al. A Novel Mitragynine Analog with Low-Efficacy Mu Opioid Receptor Agonism Displays Antinociception with Attenuated Adverse Effects. J. Med. Chem. 64, 13873-13892 (2021).
47. Wilson, L. L. et al. Characterization of CM-398, a Novel Selective Sigma-2 Receptor Ligand, as a Potential Therapeutic for Neuropathic Pain. Molecules 27, (2022).
48. Uprety, R. et al. Controlling opioid receptor functional selectivity by targeting distinct subpockets of the orthosteric site. Elife 10, 1-58 (2021)
49. Wilson, L. L. et al. Lyophilized Kratom Tea as a Therapeutic Option for Opioid Dependence. Drug Alcohol Depend. 216, 108310-108318 (2020).
50. Wilson, L. L. et al. Kratom Alkaloids, Natural and Semi-Synthetic, Show Less Physical Dependence and Ameliorate Opioid Withdrawal. Cell. Mol. Neurobiol. 41, 1131-1143 (2021).

What is claimed is:

1. A method of positively modulating μ opioid receptor (MOR) comprising contacting MOR with a compound have a structure of 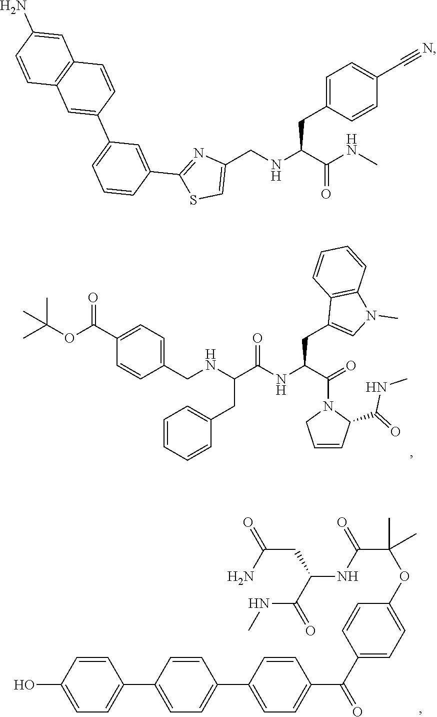 [SMILES: CNC(=O)[C@H](Cc1ccc(C#N)cc1)NCc1csc(-c2cccc(-c3ccc4cc(N)ccc4c3)c2)n1 ; CNC(=O)[C@@H]1C=CCN1C(=O)[C@H](Cc1cn(C)c2ccccc12)NC(=O)C(Cc1ccccc1)NCc1ccc(C(=O)OC(C)(C)C)cc1 ; CNC(=O)[C@H](CC(N)=O)NC(=O)C(C)(C)Oc1ccc(C(=O)c2ccc(-c3ccc(-c4ccc(O)cc4)cc3)cc2)cc1]
or a combination thereof, in an amount sufficient to positively modulate MOR.

2. The method of claim 1, wherein the compound is

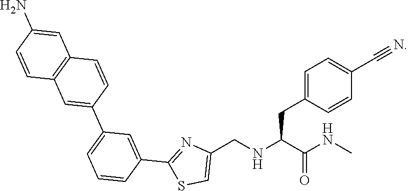 [SMILES: CNC(=O)[C@H](Cc1ccc(C#N)cc1)NCc1csc(-c2cccc(-c3ccc4cc(N)ccc4c3)c2)n1]

3. A method of positively modulating cannabinoid 1 receptor (CB1) comprising contacting CB1 with a compound having a structure 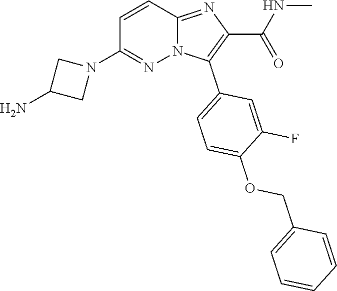 [SMILES: CNC(=O)c1nc2ccc(N3CC(N)C3)nn2c1-c1ccc(OCc2ccccc2)c(F)c1]
in an amount sufficient to positively modulate CB1.

4. A method for treating pain in a subject suffering therefrom comprising:
administering to the subject a therapeutically effective amount of a compound having a structure of 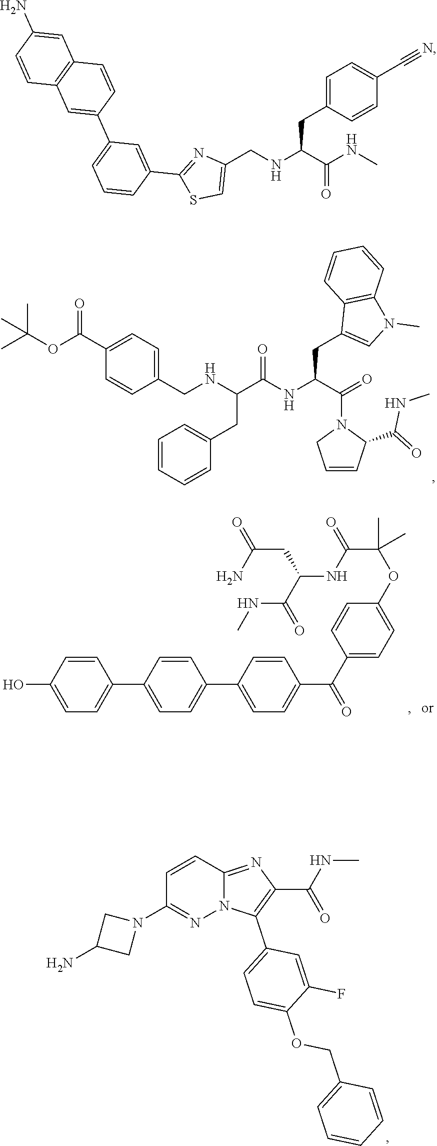 [SMILES: CNC(=O)[C@H](Cc1ccc(C#N)cc1)NCc1csc(-c2cccc(-c3ccc4cc(N)ccc4c3)c2)n1 ; CNC(=O)[C@@H]1C=CCN1C(=O)[C@H](Cc1cn(C)c2ccccc12)NC(=O)C(Cc1ccccc1)NCc1ccc(C(=O)OC(C)(C)C)cc1 ; CNC(=O)[C@H](CC(N)=O)NC(=O)C(C)(C)Oc1ccc(C(=O)c2ccc(-c3ccc(-c4ccc(O)cc4)cc3)cc2)cc1 ; CNC(=O)c1nc2ccc(N3CC(N)C3)nn2c1-c1ccc(OCc2ccccc2)c(F)c1]
or a combination thereof.

5. The method of claim 4, wherein the compound is

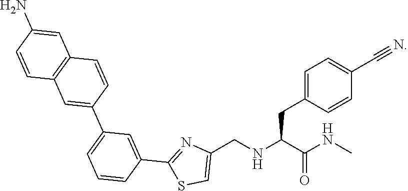 [SMILES: CNC(=O)[C@H](Cc1ccc(C#N)cc1)NCc1csc(-c2cccc(-c3ccc4cc(N)ccc4c3)c2)n1]

6. The method of claim 4, wherein the compound is

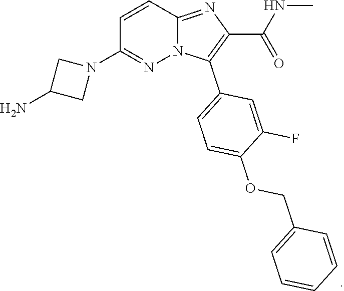 [SMILES: CNC(=O)c1nc2ccc(N3CC(N)C3)nn2c1-c1ccc(OCc2ccccc2)c(F)c1]

7. A method of inhibiting μ opioid receptor (MOR), comprising contacting MOR with compound having a structure of

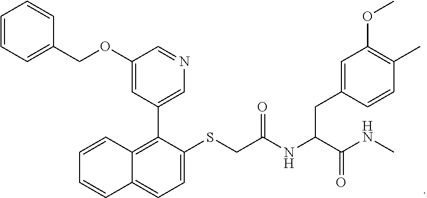 [SMILES: CNC(=O)C(Cc1ccc(C)c(OC)c1)NC(=O)CSc1ccc2ccccc2c1-c1cncc(OCc2ccccc2)c1]

8. A method for treating opioid overdose in a subject suffering therefrom comprising administering to the subject a therapeutically effective amount of a compound having a structure of

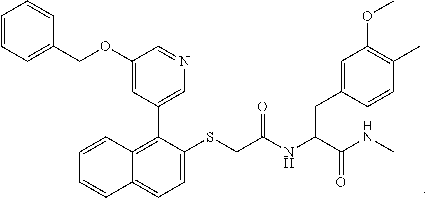 [SMILES: CNC(=O)C(Cc1ccc(C)c(OC)c1)NC(=O)CSc1ccc2ccccc2c1-c1cncc(OCc2ccccc2)c1]

9. The method of claim 8, further comprising administration of a therapeutically effective amount of naloxone either before, after, or concomitantly with administration of the compound.

10. A compound, or pharmaceutically acceptable salt thereof, wherein the compound is

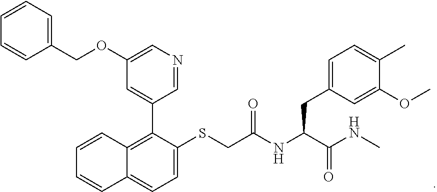 [SMILES: CNC(=O)[C@H](Cc1ccc(C)c(OC)c1)NC(=O)CSc1ccc2ccccc2c1-c1cncc(OCc2ccccc2)c1]

11. The compound, or pharmaceutically acceptable salt thereof, wherein the compound is

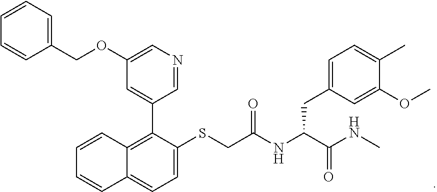 [SMILES: CNC(=O)[C@@H](Cc1ccc(C)c(OC)c1)NC(=O)CSc1ccc2ccccc2c1-c1cncc(OCc2ccccc2)c1]

12. A method for inhibiting μ opioid receptor (MOR), comprising contacting MOR with the compound of claim 10 or 11 in an amount sufficient to inhibit MOR.
